(19) United States
(12) Patent Application Publication
(10) Pub. No.: US 2026/0204348 A1
(43) Pub. Date: Jul. 16, 2026

(54) Biomolecule Fitness Inference for Variant Nomination Using Machine Learning with Directed Evolution

(71) Applicant: Genentech, Inc., South San Francisco, CA (US)

(72) Inventors: Ryan Lewis KELLY, San Mateo, CA (US); Max Walt SHEN, San Francisco, CA (US)

(21) Appl. No.: 19/447,728

(22) Filed: Jan. 13, 2026

Related U.S. Application Data
(60) Provisional application No. 63/745,229, filed on Jan. 14, 2025.

(51) Int. Cl. G16B 20/50; G16B 5/20; G16B 15/30; G16B 30/00; G16B 40/30

(52) U.S. Cl. CPC G16B 20/50; G16B 5/20; G16B 15/30; G16B 30/00; G16B 40/30

(57) ABSTRACT

In one embodiment, a method includes obtaining sequencing time-series data from a first round of directed evolution, wherein the sequencing time-series data for the first round comprises a first biomolecule frequency of a first biomolecule, the first biomolecule frequency of the first biomolecule being a non-zero frequency, obtaining sequencing time-series data from a second round of directed evolution, wherein the sequencing time-series data for the second round comprises a second biomolecule frequency of the first biomolecule, and outputting an inferred fitness score for the first biomolecule using the first biomolecule frequency and the second biomolecule frequency.

### PRIORITY

This application claims the benefit, under 35 U.S.C. § 119(e), of U.S. Provisional Patent Application No. 63/745,229, filed 14 Jan. 2025, which is incorporated herein by reference.

### TECHNICAL FIELD

This disclosure generally relates to systems and methods for inferring biomolecule fitness, and more specifically to machine-learning techniques for biomolecule selection.

### BACKGROUND

Directed evolution, with iterated mutation and human-designed selection, is a powerful approach for drug discovery, such as large molecule drug discovery. Mutation is an important part of directed evolution. Directed evolution approaches for drug discovery use genetic strategies (e.g., DNA-encoded, RNA-encoded, or phage-based) to create very large but specific libraries of molecules whose amplification is driven by the target of interest. In other words, directed evolution approaches can discover drug-like biomolecules, such as macrocycles, with novel activities of interest.

Current multiplexed target-binding candidate screening analysis systems have difficulty with the simultaneous selection of many nucleotide-containing peptide libraries for binding to a desired target due to problems such as sample-to-sample variations and data complexity. There is, therefore, a need for improved multiplexed target-binding candidate screening analysis systems and methods to help simultaneous selection of candidate binders against a desired binding target, e.g., a protein.

### SUMMARY OF PARTICULAR EMBODIMENTS

Herein is provided a system and methods for biomolecule fitness inference. Challenges exist in how to select diverse biomolecules with improved binding capabilities from directed evolution experiments. One solution is to utilize machine-learning techniques to infer biomolecule fitness based on sequencing time-series data obtained from directed evolution experiments and then select biomolecules based on the inferred fitness.

Iterated screens, such as directed evolution, can be a main approach to high-throughput affinity maturation for optimizing molecular interfaces, but the decision problem of nominating variants from an evolved population for low-throughput follow-up may remain understudied. In particular embodiments, a molecule nomination system described herein utilizes evolutionary fitness inference (EVFI) and DeepEVFI for variant nomination. EVFI and DeepEVFI are machine-learning methods that infer fitness, a variant's ability to enrich under selection pressure, from time-series sequencing data of iterated screens like directed evolution. On nine datasets spanning phage, yeast, and mRNA display, the disclosed methods outperform other methods at simulating population dynamics under held-out selection rounds. Compared to conventional variant nomination approaches used by human experts, EVFI can discover nanomolar and sub-nanomolar antibodies from yeast display one selection round earlier, and DeepEVFI can discover macrocyclic peptides from mRNA display with 10× improved binding affinity, while achieving a binder hit rate of 85%. Fitness inference can convert high-throughput DNA sequencing of evolved populations into large-scale sequence-fitness datasets which can be mined for diverse variants.

In particular embodiments, a molecule nomination system may obtain sequencing time-series data from a first round of directed evolution. The sequencing time-series data for the first round may comprise a first biomolecule frequency of a first biomolecule. The first biomolecule frequency of the first biomolecule may be a non-zero frequency. In particular embodiments, the molecule nomination system may then obtain sequencing time-series data from a second round of directed evolution. The sequencing time-series data for the second round may comprise a second biomolecule frequency of the first biomolecule. The molecule nomination system may further output an inferred fitness score for the first biomolecule using the first biomolecule frequency and the second biomolecule frequency.

Certain technical challenges exist for biomolecule fitness selection. One technical challenge may include the optimization problem induced by the flexibility to model a large variety of mutation processes. The optimization problem may include, for example, continuous optimization for fitness and initial abundances and discrete optimization for initial timepoints. One solution presented by the embodiments disclosed herein to address this challenge may be using masking during optimization to focus on fitness inference using variants that are present in the population, which can sidestep explicitly modeling when variants enter the population and their initial frequencies. Another solution presented by the embodiments disclosed herein to address this challenge may be jointly learning a sequence-to-fitness neural network for fitness inference, alongside initial abundances, using a conservative estimate of initial timepoints, which can efficiently approximate the solutions for the optimization problem. Another technical challenge may include increasing parameters to be optimized with increasingly complex datasets. The solution presented by the embodiments disclosed herein to address this challenge may be using a loss function that can be decomposed onto edges in timepoint relationship graph so that there is a constant number of inference parameters regardless of dataset complexity. Another technical challenge may include the noise and fitness uncertainty from low read counts. As disclosed herein, “read counts” refer to the number of times a variant is detected. Low read counts mean variants are detected for a number of times lower than a threshold number. The solution presented by the embodiments disclosed herein to address this challenge may be using a Dirichlet-multinomial loss to optimize the fitness inference task as the Dirichlet-multinomial loss may model a form of genetic drift, where there is random variation around each variant's growth from the previous timepoint. Another technical challenge may include training efficiency and the ability to find a good optimum in a reasonable amount of time. The solution presented by the embodiments disclosed herein to address this challenge may be a three-stage approach including obtaining a fitness vector, training the deep neural network with mean-squared-error loss on predicting the fitness vector, and performing full end-to-end training, combining count table likelihood loss with mean-squared-error loss, which can avoid optimization issues caused by the non-identifiability property by not mini batching over variants.

Certain embodiments disclosed herein may provide one or more technical advantages. A technical advantage of the embodiments may include identifying the fitness of biomolecules not in the original enrichment rounds as the molecule discovery system utilizes a deep learning model that can infer fitness for any unseen biomolecules. Another technical advantage of the embodiments may include more effective selection of discovered hits as the molecule discover system infers fitness of biomolecules that indicates biological activity and then determines discovered hits based on such biological activity. Another technical advantage of the embodiments may include the ability to discover novel and diverse variants as the molecule nomination system may identify variants with high fitness but low frequency, which may be missed through standard approaches for biomolecule discovery. Certain embodiments disclosed herein may provide none, some, or all of the above technical advantages. One or more other technical advantages may be readily apparent to one skilled in the art in view of the figures, descriptions, and claims of the present disclosure.

In particular embodiments, the techniques described herein relate to a method including, by one or more computing systems: obtaining sequencing time-series data from a first round of directed evolution, wherein the sequencing time-series data for the first round comprises a first biomolecule frequency of a first biomolecule, the first biomolecule frequency of the first biomolecule being a non-zero frequency; obtaining sequencing time-series data from a second round of directed evolution, wherein the sequencing time-series data for the second round comprises a second biomolecule frequency of the first biomolecule; and outputting an inferred fitness score for the first biomolecule using the first biomolecule frequency and the second biomolecule frequency.

In particular embodiments, the techniques described herein relate to a method, further including: accessing a biomolecule representation of the first biomolecule; processing, by a machine-learning model, the biomolecule representation of the first biomolecule the first biomolecule frequency and the second biomolecule frequency; and outputting the inferred fitness score for the first biomolecule by the machine-learning model based on the processing of the biomolecule representation of the first biomolecule.

In particular embodiments, the techniques described herein relate to a method, wherein the machine-learning model was trained using sequencing time-series data associated with biomolecule frequencies of particular biomolecules, wherein the sequencing time-series data was obtained from directed evolution of a population of biomolecules over a plurality of rounds, and wherein the population of biomolecules in each round was a unique set of biomolecules with respect to each other round.

In particular embodiments, the techniques described herein relate to a method, further including: training the machine-learning model, wherein the training includes learning inferred fitness scores for the population of biomolecules by predicting biomolecule frequencies of the population of biomolecules in the respective round given, for each biomolecule in the population of biomolecules, the biomolecule frequency of the respective biomolecule in one or more prior rounds having a non-zero frequency of the respective biomolecule.

In particular embodiments, the techniques described herein relate to a method, wherein predicting biomolecule frequencies of the population of biomolecules in the respective round given, for each biomolecule in the population of biomolecules, the biomolecule frequency of the respective biomolecule in one or more prior rounds having a non-zero frequency of the respective biomolecule includes: determining an initial round for each biomolecule where the biomolecule has a non-zero abundance; predicting an abundance associated with each biomolecule in each round after the initial round based on the non-zero abundance; and predicting biomolecule frequencies of the population of biomolecules in the respective round given predicted abundances associated with the population of biomolecules in the respective round.

In particular embodiments, the techniques described herein relate to a method, wherein the training further includes: identifying, based on the sequencing time-series data, one or more first biomolecule frequencies associated with one or more first biomolecules of the population of biomolecules that are greater than zero in a first round of the plurality of rounds; identifying, based on the sequencing time-series data, one or more second biomolecule frequencies associated with the one or more first biomolecules in a second round consecutive to the first round; predicting biomolecule frequencies associated with the one or more first biomolecules in the second round given the one or more first biomolecule frequencies; calculating a loss between the predicted biomolecule frequencies and the second biomolecule frequencies; and determining baseline fitness scores for the population of biomolecules based on the loss.

In particular embodiments, the techniques described herein relate to a method, wherein learning the inferred fitness scores for the population of biomolecules is based on the baseline fitness scores for the population of biomolecules.

In particular embodiments, the techniques described herein relate to a method, wherein learning the inferred fitness scores in the training of the machine-learning model includes optimizing a Dirichlet-multinomial loss function.

In particular embodiments, the techniques described herein relate to a method, wherein the training of the machine-learning model further includes: calculating a Dirichlet loss negative log-likelihood between the predicted biomolecule frequencies and actual biomolecule frequencies as a negative log-likelihood.

In particular embodiments, the techniques described herein relate to a method, wherein the first biomolecule is within the population of biomolecules in the plurality of rounds, wherein the first biomolecule is present in a second-to-last round of the plurality of rounds, wherein the method further includes: predicting, based on the inferred fitness score for the first biomolecule, a biomolecule frequency of the first biomolecule in a last round of the plurality of rounds.

In particular embodiments, the techniques described herein relate to a method, wherein the plurality of rounds includes at least two rounds.

In particular embodiments, the techniques described herein relate to a method, wherein the first biomolecule is outside of the population of biomolecules in the plurality of rounds.

In particular embodiments, the techniques described herein relate to a method, further including: processing a plurality of biomolecule representations associated with a plurality of respective second biomolecules by the machine-learning model to determine a plurality of inferred fitness scores for the plurality of second biomolecules, respectively; and selecting, based on the inferred fitness scores for the plurality of second biomolecules and pairwise distances between the plurality of second biomolecules, one or more diverse biomolecules from the plurality of second biomolecules, and wherein the one or more diverse biomolecules meet a predetermined criteria for selection.

In particular embodiments, the techniques described herein relate to a method, further including: processing a plurality of biomolecule representations associated with a plurality of respective second biomolecules by the machine-learning model to determine a plurality of inferred fitness scores for the plurality of second biomolecules, respectively; generating one or more clusters for the plurality of biomolecule representations based on a clustering algorithm; and selecting one or more diverse biomolecules from the plurality of second biomolecules by identifying the one or more diverse biomolecules from the one or more clusters, respectively, wherein each of the one or more diverse biomolecules is associated with a top inferred fitness score in the respective cluster.

In particular embodiments, the techniques described herein relate to a method, further including: processing a plurality of biomolecule representations associated with a plurality of respective second biomolecules by the machine-learning model to determine a plurality of inferred fitness scores for the plurality of second biomolecules, respectively; and selecting, based on the inferred fitness scores for the plurality of second biomolecules, one or more second biomolecules meeting a predetermined criteria for selection, wherein one or more of the selected second biomolecules are each associated with a low relative biomolecule frequency in a last round of the plurality of rounds.

In particular embodiments, the techniques described herein relate to a method, wherein the sequencing time-series data include DNA sequencing time-series data collected from one or more assays including one or more of a yeast display, a phage display, an mRNA display, a ribosomal display, a yeast growth, a deep mutational scan, a gene enrichment screen, or a selection on DNA encoded chemical libraries.

In particular embodiments, the techniques described herein relate to a method, wherein the sequencing time-series data include DNA sequencing time-series data collected from a yeast display, and wherein the first biomolecule includes an antibody, wherein the method further includes: determining, based on the inferred fitness score for the antibody, that a binding affinity of the antibody to receptor tyrosine kinase meets a predetermined criteria for selection.

In particular embodiments, the techniques described herein relate to a method, wherein the sequencing time-series data include DNA sequencing time-series data collected from an mRNA display, and wherein the first biomolecule includes a macrocyclic peptide, wherein the method further includes: determining, based on the inferred fitness score for the macrocyclic peptide, that a binding affinity of the macrocyclic peptide to one or more domains of receptor tyrosine kinase meets a predetermined criteria for selection.

In particular embodiments, the techniques described herein relate to a method, further including: determining, based on the inferred fitness score for the first biomolecule, that a biological activity associated with the first biomolecule meets a predetermined criteria for selection.

In particular embodiments, the techniques described herein relate to a method, wherein the inferred fitness score for the first biomolecule indicates a biological activity of the first biomolecule with respect to a target protein.

In particular embodiments, the techniques described herein relate to a method, wherein the sequencing time-series data include DNA sequencing time-series data, and wherein the first or second biomolecule frequency of the first biomolecule indicates genotype frequency.

In particular embodiments, the techniques described herein relate to one or more computer-readable non-transitory storage media embodying software that is operable when executed to: obtain sequencing time-series data from a first round of directed evolution, wherein the sequencing time-series data for the first round includes a first biomolecule frequency of a first biomolecule, the first biomolecule frequency of the first biomolecule being a non-zero frequency; obtain sequencing time-series data from a second round of directed evolution, wherein the sequencing time-series data for the second round includes a second biomolecule frequency of the first biomolecule; and output an inferred fitness score for the first biomolecule using the first biomolecule frequency and the second biomolecule frequency.

In particular embodiments, the techniques described herein relate to a system including: one or more processors; and a non-transitory memory coupled to the processors including instructions executable by the processors, the processors operable when executing the instructions to: obtain sequencing time-series data from a first round of directed evolution, wherein the sequencing time-series data for the first round includes a first biomolecule frequency of a first biomolecule, the first biomolecule frequency of the first biomolecule being a non-zero frequency; obtain sequencing time-series data from a second round of directed evolution, wherein the sequencing time-series data for the second round includes a second biomolecule frequency of the first biomolecule; and output an inferred fitness score for the first biomolecule using the first biomolecule frequency and the second biomolecule frequency.

The embodiments disclosed herein are only examples, and the scope of this disclosure is not limited to them. Particular embodiments may include all, some, or none of the components, elements, features, functions, operations, or steps of the embodiments disclosed herein. Embodiments according to the invention are in particular disclosed in the attached claims directed to a method, a storage medium, a system and a computer program product, wherein any feature mentioned in one claim category, e.g. method, can be claimed in another claim category, e.g. system, as well. The dependencies or references back in the attached claims are chosen for formal reasons only. However any subject matter resulting from a deliberate reference back to any previous claims (in particular multiple dependencies) can be claimed as well, so that any combination of claims and the features thereof are disclosed and can be claimed regardless of the dependencies chosen in the attached claims. The subject-matter which can be claimed comprises not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

### BRIEF DESCRIPTION OF THE DRAWINGS

FIG. 1A illustrates an example schematic of the decision problem of variant nomination for directed evolution or iterated screens.

FIG. 1B illustrates an example schematic of using fitness inference on variant frequency trajectories to find rising stars.

FIG. 1C illustrates example relationship between binding affinity, evolutionary fitness, and timepoint population measurements when using display assays to discover high-affinity binders.

FIG. 1D illustrates an example schematic of evolutionary fitness inference (EVFI) with zero masking.

FIG. 1E illustrates an example schematic of deep evolutionary fitness inference (DeepEVFI).

FIG. 1F illustrates an example system diagram associated with a molecule nomination system.

FIG. 2A illustrates an example v-trajectory.

FIG. 2B illustrates example noise-weighted Pearson correlation between predicted final timepoint variant enrichment from the second-to-last timepoint, compared to held-out observed enrichment.

FIG. 2C illustrates example negative log likelihood of round-over-round enrichment compared to final round enrichment.

FIG. 2D illustrates example noise-weighted Pearson correlation of round-over-round enrichment compared to final round enrichment.

FIG. 3A illustrates example variant frequency trajectories on datasets A, B, C, and D.

FIG. 3B illustrates example variant frequency trajectories on datasets F, Y-Ab, and M-MP.

FIG. 3C illustrates example normalized abundance trajectories on datasets A, B, C, and D.

FIG. 3D illustrates example normalized abundance trajectories on datasets F, Y-Ab, and M-MP.

FIG. 4A illustrates an example scatterplot of for D3b.

FIG. 4B illustrates an example scatterplot of for D3c.

FIG. 4C illustrates an example scatterplot of for both D3b and D3c.

FIG. 4D illustrates an example comparison of log EVFI inferred fitness of nominated truncated variants and the geometric mean of fold improvement on binding D3b and D3c over seed of full-length nominated variants.

FIG. 4E illustrates example frequency trajectories of variants.

FIG. 5A illustrates an example diagram depicting SPR outcomes for nominated variant sets on macrocyclic peptides binding to TEAD.

FIG. 5B illustrates example TEAD KD for variants.

FIG. 5C illustrates example trajectories of DeepEVFI-nominated variants in M-MP dataset.

FIG. 6A illustrates example UMAP plots of sequences.

FIG. 6B illustrates example UMAP plots of sequences.

FIG. 6C illustrates example Pareto frontier of nomination sets with 96 variants optimizing average log fitness or average pairwise distance compared to nomination sets by sampling 96 variants by final timepoint frequency.

FIGS. 7A-7B illustrate example sorted frequencies of variants in final timepoints.

FIG. 8 illustrates an example method for biomolecule fitness inference.

FIG. 9 illustrates an example computer system.

### DESCRIPTION OF EXAMPLE EMBODIMENTS

### Introduction

Directed evolution and iterated screening, performed using yeast surface display, phage display, and mRNA display, can be powerful methods for protein engineering and drug discovery, in which a diverse population can be optimized for desired properties over multiple rounds of selection, replication, and potentially mutation. The ability to link desired biological activity to survival rate may enable engineering biomolecules for a wide variety of properties. In vitro display assays can explore substantial sequence space over time, with library sizes up to 109 in yeast, 1011 in phage, and 1013 for mRNA display. Mutations can arise from many sources, can be difficult to eliminate, and can improve exploration of fitness landscapes. Timepoint populations from multiple rounds of directed evolution can contain useful genotypic diversity, with rich dynamics of competing variants emerging, increasing in frequency, and declining.

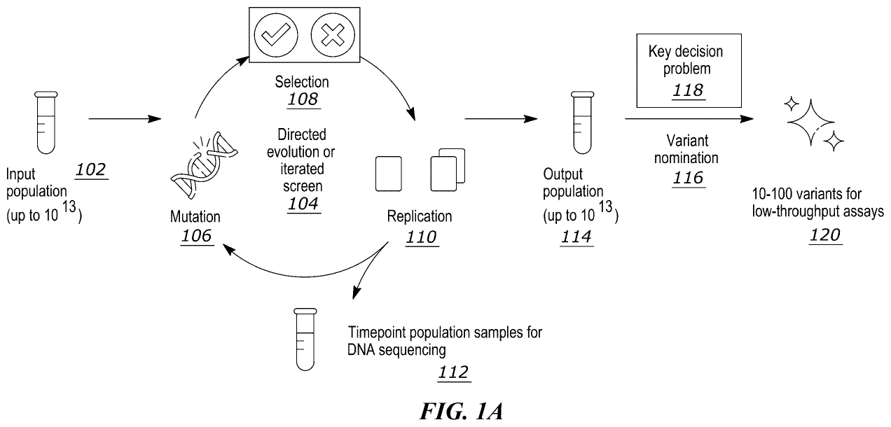

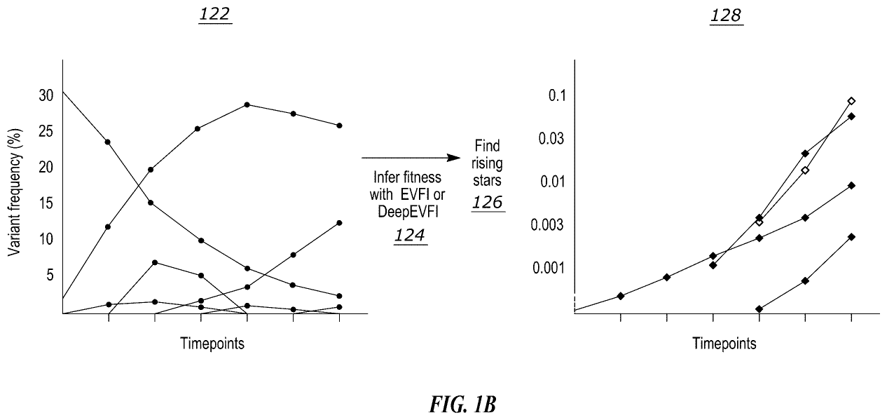

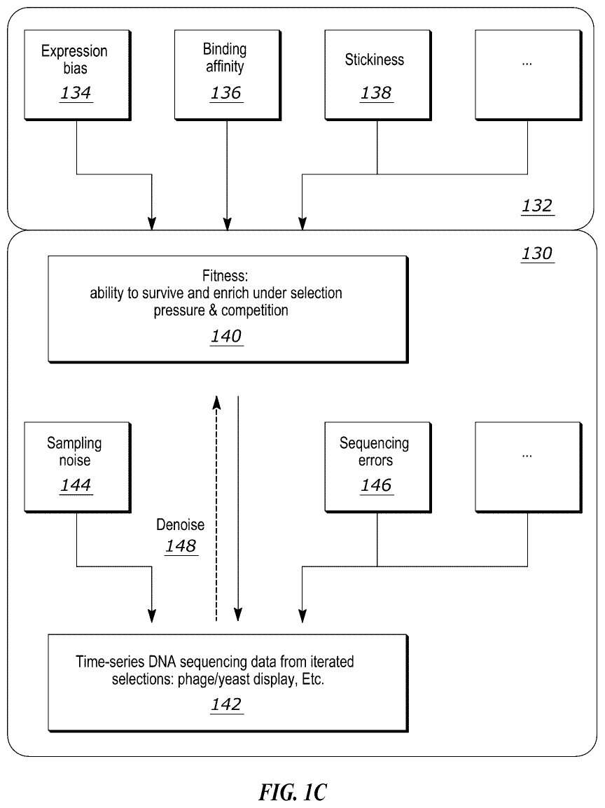

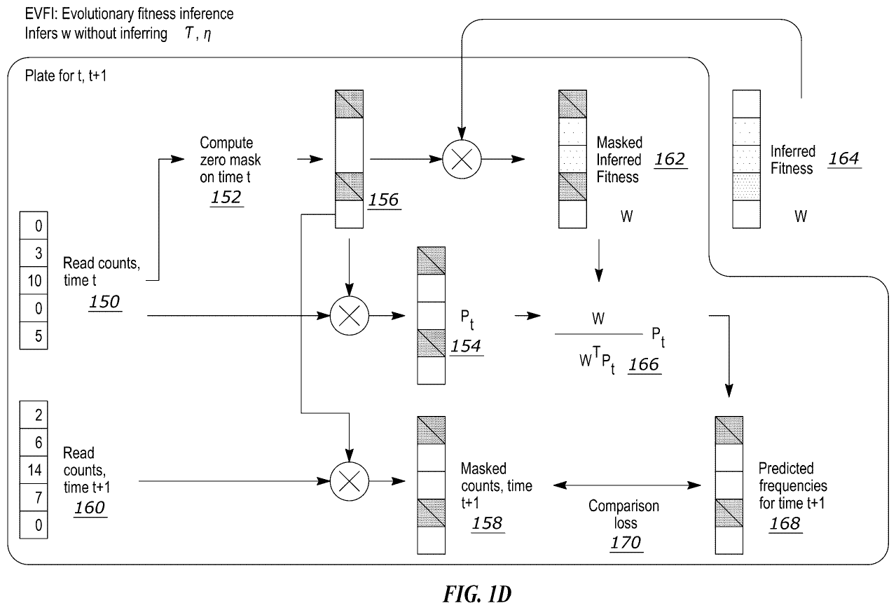

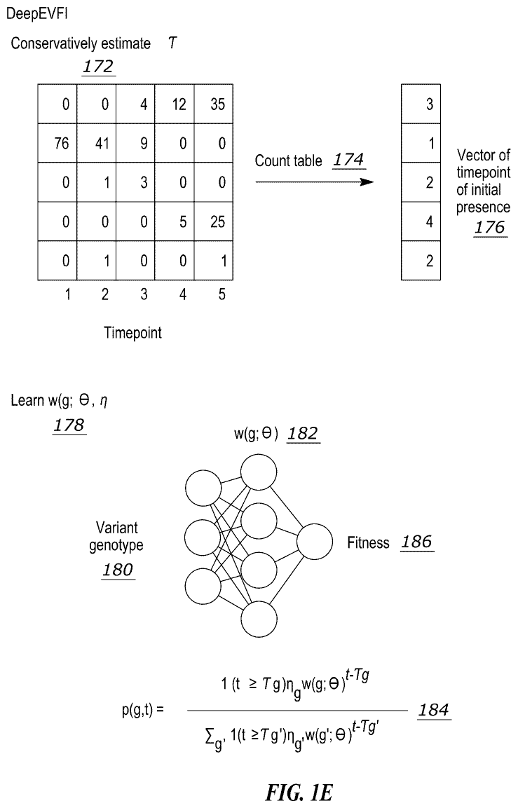

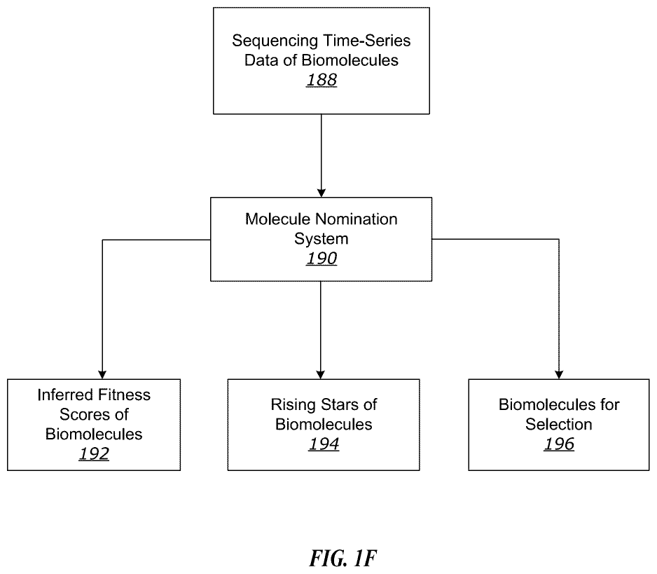

Directed evolution can produce an evolved population with substantial diversity, but researchers may be interested in nominating a small number of variants, dozens to hundreds, for low-throughput follow-up study (FIG. 1A). This decision problem of variant nomination thus can play a key role in campaign success, yet little work has studied whether the full history of evolutionary dynamics could be used to improve variant nomination for in vitro directed evolution. The overwhelmingly dominant approach to variant nomination in conventional yeast, phage, and mRNA display literature may use final timepoint frequencies, and it is unclear how optimal this approach is.

The embodiments disclosed herein consider modeling fitness without functional data, primarily using measurements of variant frequencies from the history of a directed evolution campaign. Two-timepoint enrichment analysis has been used for deep mutational scanning and ranking variants from screens, and generative models have been trained on variant frequencies and on two-timepoint enrichment, but these approaches may not use all the data available in an evolutionary history with three or more time-points. Binder discovery methods may compare negative and positive sorts, but these branching populations may not reside on a single history of evolution.

Evolutionary dynamics have been modeled in bacterial growth, cancer, yeast growth, and continuous evolution. ACIDES (i.e., a conventional method) and Enrich2 (i.e., another conventional method) may model enrichment from three or more timepoints from in vitro iterated selections but may be not designed for data with mutations. AMaLa (i.e., another conventional method) may model iterated screens under a restrictive mutation model centered on a wildtype sequence. No methods may support the fully general setting of directed evolution, where mutations may or may not occur, and the initial population can start from a single wildtype sequence or start from a highly diverse population lacking a meaningful wildtype. Furthermore, no comparable methods have been experimentally evaluated for variant nomination with functional assays.

EVFI and DeepEVFI are machine learning methods for efficiently and approximately solving a challenging optimization problem induced by a flexible model of mutation, which may enable modeling a broad range of time-series sequencing data such as in vitro directed evolution from yeast, phage, and mRNA display. As used herein, a flexible model of mutation may refer to a computational model that allows for a wide range of mutation types and effects. This disclosure shows that deep learning can improve fitness inference on datasets with three or more timepoints by more accurately predict population changes in held-out selection rounds, building on work that augments two-timepoint enrichment analysis with deep learning. The embodiments disclosed herein discover that “rising stars”—high-fitness, low-frequency variants that could dominate the population given further selection rounds—can be common in yeast, phage, and mRNA display data. Finally, this disclosure shows that fitness inference can improve variant nomination compared to conventional methods used by human experts. In yeast display on antibodies, the embodiments disclosed herein achieve a binder hit rate of 80%, outperforming variants nominated by a human expert using the same number of selection rounds, and matching performance to human-expert variants nominated from an additional selection round. In mRNA display on macrocycles, the embodiments disclosed herein achieve a binder hit rate of 90%, and 92% of the binder's match or improve affinity of the best human-expert binder. Fitness inference can enable processing high-throughput DNA sequencing of evolved populations into large-scale sequence-fitness datasets, which can be mined for diverse variants, and used to train sequence-fitness models.

For the readability of this disclosure, Table 1 below lists all notations described herein.

TABLE 1
Notations described in this disclosure.
Variables from Observed Data

| | |
|---|---|
| G | Total number of unique genotype variants in a dataset; |
| | a positive integer, indexed by g. |
| T | Total number of timepoints in a dataset; |
| | a positive integer, indexed by t. |
| c_{g,t} | The number of DNA sequencing reads for variant |
| | g at timepoint t; a non-negative integer. |
| p_{g,t} | The fractional proportion of variant g at |
| | timepoint t among all DNA sequencing |
| | reads at timepoint t. Defined as c_{g,t}/Σ_{g′}c_{g′,t}. |
| | A real number between 0 and 1. |

Unobserved Variables

| | |
|---|---|
| n_{g,t} | The absolute quantity of variant g at timepoint t, |
| | such as number of physical molecules. |
| | This is not measurable with DNA sequencing; |
| | a non-negative real number. |
| ω_{g} | Absolute fitness of variant g. A non-negative |
| | real number. Not identifiable from data; can only |
| | be identified up to an unknown multiplicative factor. |

Inference Variables

| | |
|---|---|
| w_{g} | Inferred fitness, or relative fitness, of variant g. |
| | A non-negative real number. |
| τ_{g} | The timepoint when variant g first enters |
| | the population and has non-zero |
| | quantity; a positive integer. |
| η_{g} | The absolute quantity of variant g at its |
| | initial timepoint τ_{g}; a non-negative real |
| | number. |
| φ_{g} | The fractional proportion of variant |
| | g at its initial timepoint τ_{g}. Defined as |
| | η_{g}/Σ_{g′}n_{g′,τg}. A real number between 0 and 1. |

Vectors and Matrices

| | |
|---|---|
| ω | Absolute fitness vector with length G, with elements ω_{g}. |
| w | Inferred fitness, or relative fitness, |
| | vector with length G, with elements ω_{g}. |
| Pₜ | Vector of observed variant frequencies |
| | with length G, with elements p_{g,t}. |
| τ | Vector of initial timepoints with length G, with elements τ_{g}. |
| η | Vector of absolute variant quantities at their |
| | initial timepoints, with length G, with elements τ_{g}· |
| φ | Vector of fractional variant proportions |
| | at their initial timepoints, with length G, |
| | with elements τ_{g}· |
| Cₜ | Vector of time-series DNA sequencing |
| | data at timepoint t with length G, with |
| | elements c_{g,t}. |
| C | Matrix of time-series DNA sequencing data |
| | with shape G × T with elements c_{g,t}. |

### Data Generative Process and Problem Statement

This section states the model of the data generative process and the inference problem for the embodiments disclosed herein.

### Model of Data Generative Process

The embodiments disclosed herein consider a process of wet lab directed evolution, where a population of genotype variants undergo iterated rounds of selection, replication, and potentially mutation. In particular embodiments, the plurality of rounds may comprise at least two rounds. Timepoint samples of the population are taken for DNA sequencing. For T total timepoints and G total unique genotype variants, this process generates a matrix C, which we call a “count table”, with entries c_{g,t} denoting the non-zero integer number of DNA sequencing reads for variant g at timepoint t (see Table 1).

Particular embodiments model growth in each round of directed evolution using a simple model of asexual natural selection, where each variant g with absolute quantity n_{g,t} (e.g., absolute abundance of number of molecules, or copies) grows at a rate over time according to its absolute fitness ω_{g}, a non-negative real number:

$${n}_{g , t + 1} = {ω}_{g} {n}_{g , t} \qquad ( 1 )$$

Assuming that variant g enters the population at most once, at initial timepoint τ_{g}, with absolute quantity η_{g}, we have:

$${n}_{g , t} = \{ \begin{matrix}0 , & t < {τ}_{g} \\ {η}_{g} & t , = {τ}_{g} \\ {ω}_{g}^{( t - {τ}_{g} )} {η}_{g} , & t > {τ}_{g}\end{matrix} \qquad ( 2 )$$

This disclosure defines the population frequency of a variant g at timepoint t as:

$${p}_{g , t} {=}^{△} \frac{{n}_{g , t}}{{\sum }_{j} {n}_{j , t}} \qquad ( 3 )$$

By algebraic manipulation, equation 1 induces non-linear dynamics on variant frequencies (See, property 1 for proof in later sections):

$${p}_{g , t + 1} = \frac{{ω}_{g}}{{\sum }_{j} {ω}_{j} {p}_{j , t}} {p}_{g , t} \qquad ( 4 )$$

Finally, particular embodiments model:

$${C}_{t} ∼ Multinomial ( p = {p}_{t} , N = {\sum }_{{g}^{′}} {c}_{{g}^{′} , t} ) \qquad ( 5 )$$

where c_{g,t} is the number of DNA sequencing reads for variant g at timepoint t, Cₜ is the vectorized version with length G, and pₜ is the vector of p_{g,t}.

### Inference Problem

Given a DNA sequencing count table C with shape G×T, this disclosure aims to infer three G-dimensional vectors: the absolute fitness vector ω, and initial timepoints τ and abundances η.

However, ω may be not identifiable from measurements of variant frequencies, even if sequencing depth or number of timepoints approach infinity. ω may be only identifiable up to an unknown multiplicative factor (See, Property 2 in Subsection of Mathematical Properties of Evolutionary Fitness Inference in Section of Methods). This may be because variant frequency dynamics in equation (4) are invariant to rescaling ω by any positive real number c.

As a result, this disclosure sets up the inference problem as:

Problem statement. Given C, infer initial timepoints τ, initial abundances η, and relative fitness w¬cω where c>0 is unknown.

This inference problem can be a challenging mixed-integer optimization problem because it includes continuous optimization for w, η and discrete optimization for initial timepoints τ. In principle, as initial timepoints for different genotype variants are independent, τ can take on up to T^{G} different values. For typical values T=6, G=30000, one may have T^{G}=2.8×10²³³. For this reason, particular embodiments developed efficient approximate methods.

### Results

Evolutionary Fitness Inference from Time-Series Data

FIG. 1A illustrates an example schematic of the decision problem of variant nomination for directed evolution or iterated screens. Input population 102 (up to 10¹³) may undergo directed evolution or iterated screen 104. In particular embodiments, directed evolution or iterated screen 104 may comprise mutation 106, selection 108, and replication 110. Timepoint population samples for DNA sequencing 112 may be collected from directed evolution or iterated screen 104. Output population 114 (up to 10¹³) may be obtained from directed evolution or iterated screen 104. Variant nomination 116 may be performed on output population 114, which can be a key decision problem 118. With variant nomination 116, 10-100 variants for low-throughput assays 120 may be nominated.

FIG. 1B illustrates an example schematic of using fitness inference on variant frequency trajectories to find rising stars. The left subfigure 122 shows variants with respect to timepoints and variant frequency (%). From these variants, the molecule nomination system may infer fitness with EVFI or DeepEVFI at operation 124 to find rising stars at operation 126. The right subfigure 128 shows the rising stars with respect to timepoints and variant frequency (%).

FIG. 1C illustrates example relationship between binding affinity, evolutionary fitness, and timepoint population measurements when using display assays to discover high-affinity binders. Box 130 indicates the general scope of EVFI as a computational method, while box 132 indicates factors dependent on experimental design and choice of assay, such as phage display, mRNA display, yeast display, etc. With target biological activity and nuisance factors such as expression bias 134, binding affinity 136, stickiness 138, etc., EVFI 130 may infer fitness 140, which indicates ability to survive and enrich under selection pressure and competition. EVFI 130 may infer fitness based on time-series DNA sequencing data from iterated selections (phage/yeast display, etc.) 142. As sampling noise 144 and sequencing errors 146 may be introduced into the time-series DNA sequencing data 142, the molecule nomination system may perform denoise 148.

FIG. 1D illustrates an example schematic of evolutionary fitness inference (EVFI) with zero masking. The rounded plate denotes variables that depend on t,t+1. The symbol ⊗ indicates elementwise multiplication. Based on read counts 150 at time t, the molecule nomination system may compute zero mask on time t at operation 152. The molecule nomination system may generate pₜ 154 based on elementwise multiplication between read counts 150 at t and zero-masked counts 156 at t. The molecule nomination system may generate masked counts 158 at t+1 based on elementwise multiplication between read counts 160 at time t+1 and zero-masked counts 156 at t. The molecule nomination system may generate masked inferred fitness 162 based on elementwise multiplicate between inferred fitness 164 and zero-masked counts 156 at t. Based on

$${p}_{t} \to \frac{w}{{w}^{T} {p}_{t}} {p}_{t} 166 ,$$

the molecule nominations system may determine predicted frequencies 168 for time t+1. The molecule nomination system may further determine comparison loss 170 between predicted frequencies 168 for time t+1 and masked counts 158 at t+1.

FIG. 1E illustrates an example schematic of deep evolutionary fitness inference (DeepEVFI). At operation 172, the molecule nomination system may conservatively estimate r. Based on a countable 174, the molecule nomination system may determine vector of timepoint of initial presence 176. At operation 178, the molecule nomination system may learn w(g; θ) and q. Based on variant genotype 180, the molecule nomination system may optimize w(g; θ) 182 using equation 184 to infer fitness 186.

FIG. 1F illustrates an example system diagram associated with a molecule nomination system. In particular embodiments, the molecule nomination system 190 may obtain sequencing time-series data of biomolecules 188. Based on the sequencing time-series data of biomolecules 188, the molecule nomination system 190 may determine inferred fitness scores 192 of the biomolecules. The molecule nomination system 190 may also identify rising stars 194 of the biomolecules among the biomolecules. As disclosed herein, rising stars refer to high-fitness, low-frequency biomolecules. The molecule system 190 may further determine biomolecules for selection 196 based on the inferred fitness scores 192 of biomolecules.

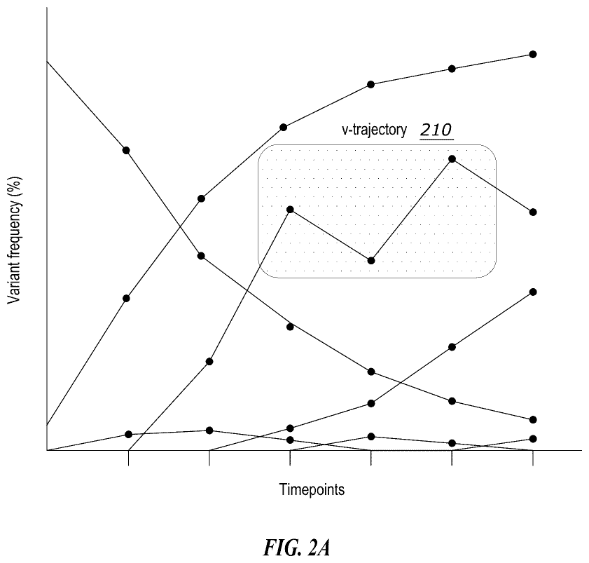

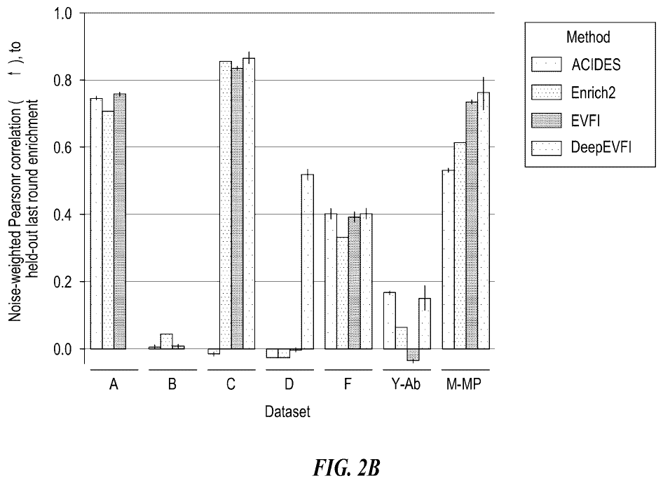

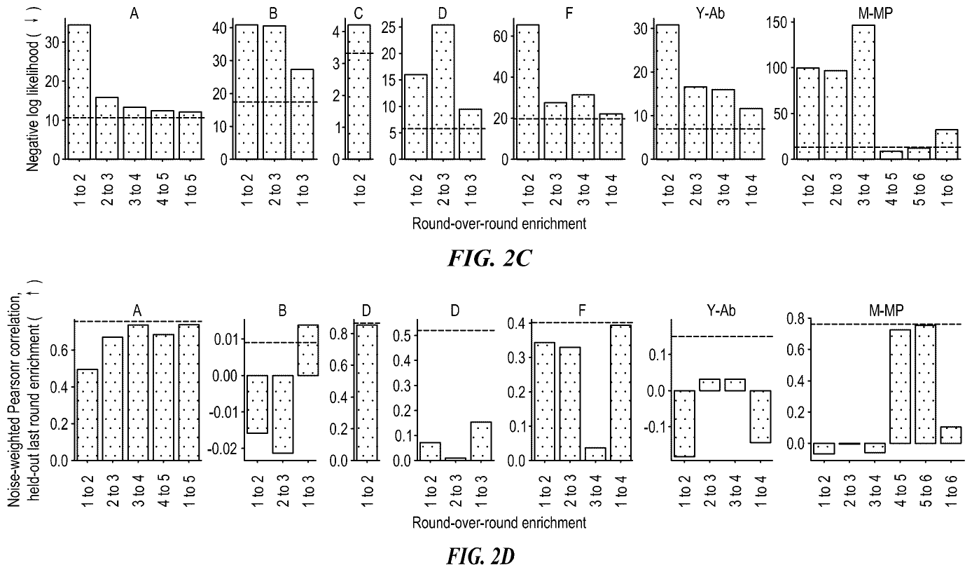

The embodiments disclosed herein consider the problem of fitness inference from T timepoints of variant frequency measurements of a population of G variants undergoing iterated rounds of selection, replication, and possibly mutation. A variant's fitness may be a positive number representing its ability to survive and grow under selection pressure and competition, due in part to a desired biological activity such as binding affinity (FIGS. 2A-2D). In particular embodiments, the inferred fitness score for the first biomolecule may indicate a biological activity of the first biomolecule with respect to a target protein. The molecule nominations system may further determine, based on the inferred fitness score for the first biomolecule, that a biological activity associated with the first biomolecule meets a predetermined criteria for selection. Time-series DNA sequencing data can be collected from many assays including yeast display, phage display, mRNA display, ribosomal display, yeast growth, deep mutational scans, gene enrichment screens, and selections on DNA encoded chemical libraries. In particular embodiments, the sequencing time-series data may comprise DNA sequencing time-series data and the first or second biomolecule frequency of the first biomolecule may indicate genotype frequency.

The embodiments disclosed herein consider a data generative process where each unique variant g has three parameters: τ_{g}, the initial timepoint it enters the population with abundance η_{g}, and ω_{g}, its absolute fitness. Even with infinite noiseless data, ω may be only identifiable up to an unknown multiplicative factor (the “non-identifiability property”), so particular embodiments instead infer w=cω where c is unknown. Inferring fitness may depend on inferring τ and η, but both may be underspecified with typical datasets, and τ can be a discrete vector with up to T^{G} different values, exceeding 10²⁰⁰ for typical values T=6, G=30000.

This challenging optimization problem may be the trade-off for the flexibility to model a large variety of mutation processes. Later in this disclosure, particular embodiments estimate mutation rates in common directed evolution platforms. Mutations arise from diverse sources, can be difficult to fully anticipate or eliminate, and can occur at roughly 10⁻³ mutations per construct (m/c) per timepoint in phage and mRNA display, and 10⁻⁶ m/c per timepoint in yeast display. The disclosed model's benefit may include its applicability to a wide variety of data, including data with mutation mechanisms that are difficult to characterize, partially or completely unknown, and data where the existence or absence of mutations is unclear.

EVFI and DeepEVFI can be efficient approximate solutions for fitness inference. EVFI can infer fitness while sidestepping explicit inference of r and q using a zero-masking approach on consecutive timepoint pairs (FIG. 1D). Using masking during optimization to focus on fitness inference using variants that are present in the population may be an effective solution for addressing the technical challenge of the optimization problem induced by the flexibility to model a large variety of mutation processes, as making can sidestep explicitly modeling when variants enter the population and their initial frequencies.

DeepEVFI can jointly learn a sequence-to-fitness neural network for fitness inference, alongside η, using a conservative estimate of τ (FIG. 1E). In particular embodiments, the molecule nomination system 190 may train the machine-learning model (e.g., DeepEVFI). The training may comprise learning inferred fitness scores for the population of biomolecules by predicting biomolecule frequencies of the population of biomolecules in the respective round given, for each biomolecule in the population of biomolecules, the biomolecule frequency of the respective biomolecule in one or more prior rounds having a non-zero frequency of the respective biomolecule. In particular embodiments, predicting biomolecule frequencies of the population of biomolecules in the respective round given, for each biomolecule in the population of biomolecules, the biomolecule frequency of the respective biomolecule in one or more prior rounds having a non-zero frequency of the respective biomolecule may comprise determining an initial round for each biomolecule where the biomolecule has a non-zero abundance, predicting an abundance associated with each biomolecule in each round after the initial round based on the non-zero abundance, and predicting biomolecule frequencies of the population of biomolecules in the respective round given predicted abundances associated with the population of biomolecules in the respective round.

Jointly learning a sequence-to-fitness neural network for fitness inference, alongside initial abundances, using a conservative estimate of initial timepoints may be another effective solution for addressing the technical challenge of the optimization problem induced by the flexibility to model a large variety of mutation processes, as the joint learning of the sequence-to-fitness neural network can efficiently approximate the solutions for the optimization problem. Both methods can optimize data likelihood under noise models which can account for genetic drift and sampling noise. Learning a sequence-to-fitness neural network may improve fitness inference, especially for low-evidence variants with low read counts, by sharing information from “similar” genotypes. This disclosure further discusses EVFI and DeepEVFI, the relationship between enrichment and fitness, and proves a theorem on how two fitness or enrichment scores, on different unknown scales, can be compared to each other, despite the non-identifiability property, alongside other details in later sections.

### Predicting Held-Out Selection Timepoints

The embodiments disclosed herein study seven public datasets of time-series DNA sequencing data of populations undergoing multiple selection rounds, spanning optimization of human protein domains, antibodies, adeno-associated virus (AAV), snoRNAs, and peptides, using methods including phage display, yeast two-hybrid, yeast growth, and in vivo directed evolution for AAV transduction. This disclosure denotes these datasets, previously studied by ACIDES, by letters A-G. In addition, the embodiments disclosed herein study and release two new datasets: a yeast surface display campaign optimizing antibodies for FGFR1 binding with six timepoints (denoted Y-Ab), and an mRNA display campaign optimizing macrocyclic peptides for TEAD binding with seven timepoints (M-MP). Table 1 describes the nine datasets. Overall, five datasets optimize variants for binding affinity to a range of targets including DNA, peptides, and proteins with therapeutic relevance.

TABLE 1
Description of nine datasets studied in the embodiments disclosed herein.

| | | | | | Unique | |
|---|---|---|---|---|---|---|
| | | | Biological | | variants after | |
| Name | Assay | Variants | activity | Timepoints | preprocessing | Total read-count |
| A | Phage display | BRCA1 | Ligase | 6 | 34,718 | 49,970,411 |
| | | | activity | | | |
| B | Yeast two- | BRCA1 | Binding | 4 | 26,743 | 52,514,178 |
| | hybrid | | affinity | | | |
| C | Phage Display | Antibody | Binding | 3 | 28,632 | 317,418 |
| | | | affinity | | | |
| D | Phage Display | hYAP65 | Binding | 4 | 470,312 | 19,991,571 |
| | | | affinity | | | |
| E | In vivo AAV | AAV gene | Transduction | 6 | 5,087 | 102,867,307 |
| | transduction | | efficacy | | | |
| F | Yeast growth | U3 snoRNA | snoRNA | 5 | 23,856 | 41,946,453 |
| | | | activity | | | |
| G | In vivo AAV | Peptide | Transduction | 5 | 576 | 30,761,962 |
| | transduction | | efficacy | | | |
| Y-Ab | Yeast display | Antibody | Binding | 6 | 27,843 | 727,072 |
| | | | affinity | | | |
| M-MP | mRNA display | Macrocyclic | Binding | 7 | 25,938 | 13,618,956 |
| | | peptide | affinity | | | |

The embodiments disclosed herein compare EVFI to ACIDES and Enrich2, representative and general methods for modeling enrichment over three or more timepoints. These methods, and the disclose method herein, assume variants have a time-invariant growth rate. However, conventional work has not characterized whether real-world datasets conform to these model assumptions. The embodiments disclosed herein introduce a statistical measure to evaluate whether datasets violate these assumptions. In a later section, this disclosure proves that under these assumptions, a “v-trajectory” cannot occur, which is a timepoint triplet where a variant decreases, then increases in frequency, with a ‘V’ shape (FIG. 2A). FIG. 2A illustrates an example v-trajectory 210. The example v-trajectory 210 is illustrated with respect to variant frequency (%) and timepoints. The embodiments disclosed herein compute the probability of a v-trajectory under binomial sampling noise, and score datasets using the total frequency of variants with a v-trajectory with high confidence (>95% probability).

On nine datasets, the two in vivo AAV datasets have serious violations of assumed dynamics with up to 47.4% and 54.5% of the population containing v-trajectories with high confidence (Table 2). Yeast growth and yeast two-hybrid campaigns had mild v-trajectory violations between 10%-15%, while yeast, phage, and mRNA display campaigns had 0%-10%. This analysis evaluating a dataset's internal consistency may provide a principled framework for deciding whether fitness inference methods can be meaningfully applied to a real-world dataset. Following this, this disclosure excludes in vivo AAV selection datasets E and G, which were previously studied with ACIDES, leaving seven datasets for further analysis.

TABLE 2
Percent of population comprised of variants with a v-trajectory
with high confidence, indicating that the real-world
dataset has violations of model assumptions.

| | | Percentage of population | | | | | | |
|---|---|---|---|---|---|---|---|---|
| | | with a v-trajectory | | | | | | |
| Timepoint | | 1 | 2 | 3 | 4 | 5 | 6 | 7 |
| Phage display | A | 7.3 | 6.9 | 6.7 | 6.7 | 6.4 | 6.4 | |
| | C | 0.03 | 0.00 | 0.04 | | | | |
| | D | 4.1 | 3.6 | 4.2 | 3.8 | | | |
| Yeast display | Y-Ab | 0.1 | 0.8 | 2.1 | 0.8 | 2.3 | | |
| Yeast growth | F | 10.0 | 12.5 | 13.2 | 12.5 | 14.2 | | |
| Yeast | B | 12.6 | 11.0 | 9.5 | 11.7 | | | |
| two-hybrid | | | | | | | | |
| mRNA display | M-MP | 3.2 | 2.0 | 1.1 | 8.2 | 4.7 | 4.9 | 3.8 |
| AAV | E | 40.4 | 20.8 | 47.4 | 35.8 | 14.8 | 15.6 | |
| | G | 28.7 | 29.2 | 54.5 | 2.5 | 10.3 | | |

The embodiments disclosed herein fit or train each method while holding out the last timepoint and evaluate performance in predicting held-out final timepoint data of variants present in the second-to-last timepoint using inferred variant growth rates. In other words, the first biomolecule may be outside of the population of biomolecules in the plurality of rounds. This evaluation can test each method's ability to infer fitness scores that accurately capture growth advantages under selection pressure and competition.

Particular embodiments first compared EVFI to ACIDES and Enrich2, as these methods may not use genotype information. EVFI performed best, achieving 2.1× improved likelihood than ACIDES (geometric mean across datasets), and 4.5× improved likelihood over Enrich2 (Table 3). EVFI achieved the best likelihood on four out of seven datasets, and otherwise performed closely to the best method ACIDES and Enrich2 had inconsistent performance: good on some datasets, but very poor on others like A (10.63 for EVFI, vs. 193.01 for Enrich2) and D (19.25 for EVFI, vs. 144.83 for ACIDES). Taken together, EVFI performed strongest among the three methods that may not use variant genotype information on seven datasets and performed reliably across a variety of iterated selection campaigns spanning phage display, yeast growth, yeast display, and mRNA display.

TABLE 3
Negative log likelihood (bits per variant; lower value is better)
comparing predicted final timepoint variant frequencies to held-
out data, reported as mean plus-or-minus standard deviation over
five training replicates. No results reported for DeepEVFI on
datasets A and B because they lack genotype information.

| Dataset | Held-out last-round data, negative log likelihood (↓) | | | |
|---|---|---|---|---|
| A | n/a | 10.63 ± 0.00 | 11.45 ± 0.00 | 193.01 ± 0.00 |
| B | n/a | 17.43 ± 0.00 | 17.42 ± 0.01 | 23.24 ± 0.00 |
| C | 3.33 ± 0.09 | 4.70 ± 0.02 | 43.30 ± 0.28 | 4.20 ± 0.00 |
| D | 5.76 ± 0.14 | 19.25 ± 0.01 | 144.83 ± 0.00 | 81.15 ± 0.00 |
| F | 19.77 ± 0.06 | 22.53 ± 0.00 | 19.71 ± 0.00 | 83.44 ± 0.00 |
| Y-Ab | 12.95 ± 0.38 | 26.74 ± 0.10 | 40.32 ± 0.09 | 21.20 ± 0.00 |
| M-MP | 6.94 ± 0.14 | 7.55 ± 0.10 | 14.68 ± 0.00 | 986.62 ± 0.00 |
| | DeepEVFI | EVFI | ACIDES | Enrich2 |

On five datasets with genotype data, DeepEVFI achieved the strongest likelihood performance, improving 1.6× over EVFI, 4.6× over ACIDES, and 7.1× over Enrich2 (geometric mean across datasets). FIG. 2B illustrates example noise-weighted Pearson correlation between predicted final timepoint variant enrichment from the second-to-last timepoint, compared to held-out observed enrichment. Bars and error bars plot mean and standard deviation over five training replicates. No results reported for DeepEVFI on datasets A and B because they lack genotype information. In all five datasets, DeepEVFI achieves the strongest noise-weighted correlation to held-out enrichment, or ties with the best performing method within one standard deviation range across training replicates.

Fitness inference may use data from all timepoints, in contrast to round-over-round enrichment, where it can be unclear which two timepoints to use among T (T−1) pairs. Performance may vary widely by timepoints used to compute enrichment, and no single choice may be best for all seven datasets. By using all time-point data, fitness inference bypasses timepoint choice for enrichment analysis and can achieve better performance. FIG. 2C illustrates example negative log likelihood of round-over-round enrichment compared to final round enrichment. FIG. 2D illustrates example noise-weighted Pearson correlation of round-over-round enrichment compared to final round enrichment. In FIGS. 2C-2D, blue line indicates EVFI or DeepEVFI. EVFI and DeepEVFI matches or exceeds the best performance of commonly used round-over-round enrichments including consecutive timepoint pairs, and last-over-first (FIGS. 2C-2D). By using all time-point data, fitness inference can bypass timepoint choice for enrichment analysis and can achieve better performance.

As shown by the evaluation above, a technical advantage of the embodiments may include identifying the fitness of biomolecules not in the original rounds as the molecule discover system utilizes a deep learning model that can infer fitness for any unseen biomolecules.

### Rising Stars are Common and Overlooked

The conventional variant nomination approach in directed evolution may sample the final timepoint population. For fitness inference to improve on this, there should exist higher fitness variants unlikely to be sampled from the final timepoint due to low frequency, which this disclosure terms rising stars. However, the extent to which rising stars exist in yeast, phage, and mRNA display has received little systematic investigation.

In particular embodiments, the molecule nomination system 190 may process a plurality of biomolecule representations associated with a plurality of respective second biomolecules by the machine-learning model to determine a plurality of inferred fitness scores for the plurality of second biomolecules, respectively. The nomination system may further select, based on the inferred fitness scores for the plurality of second biomolecules, one or more second biomolecules meeting a predetermined criteria for selection. One or more of the selected second biomolecules may be each associated with a low relative biomolecule frequency in a last round of the plurality of rounds.

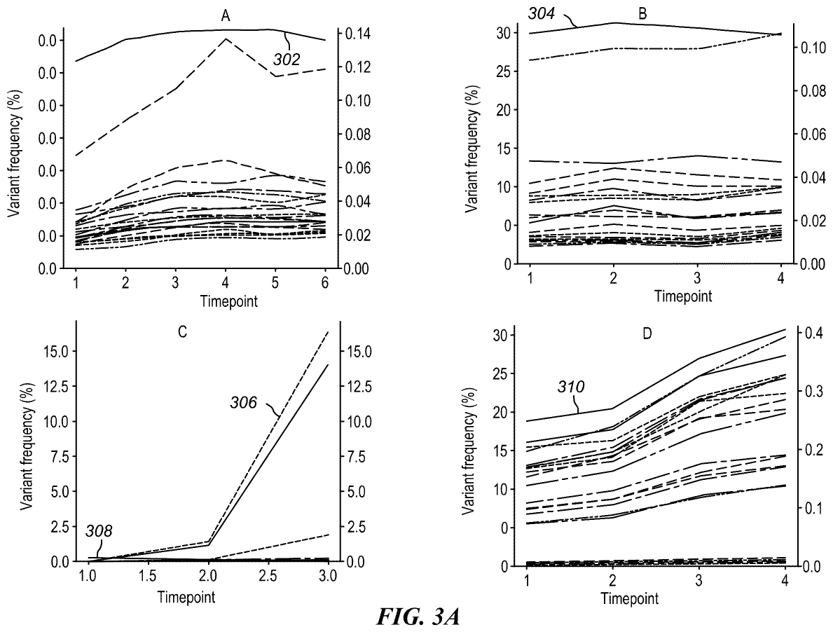

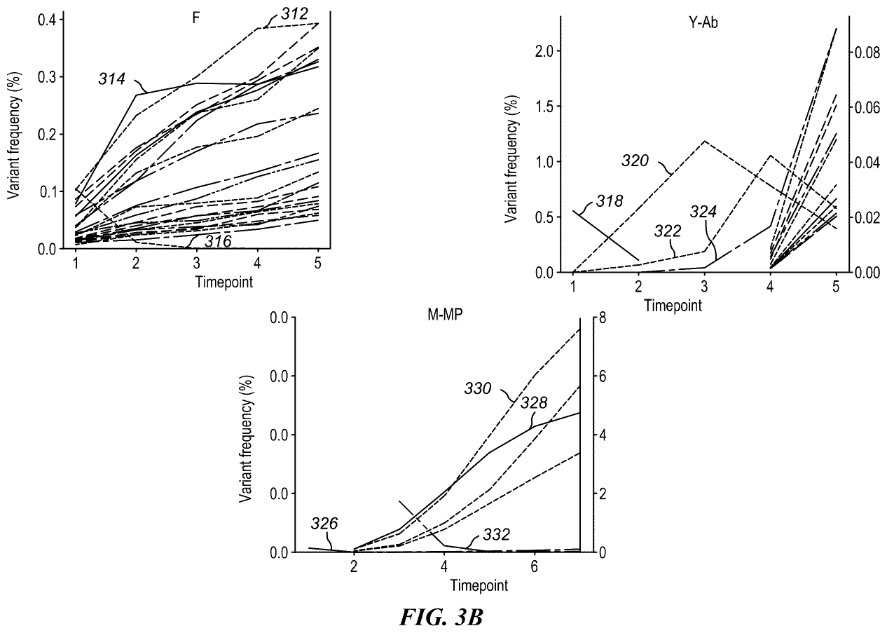

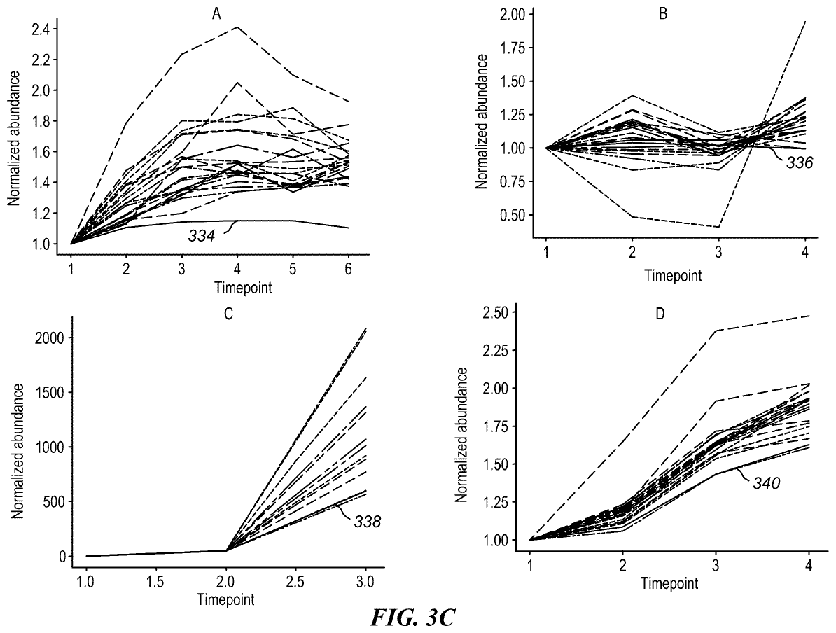

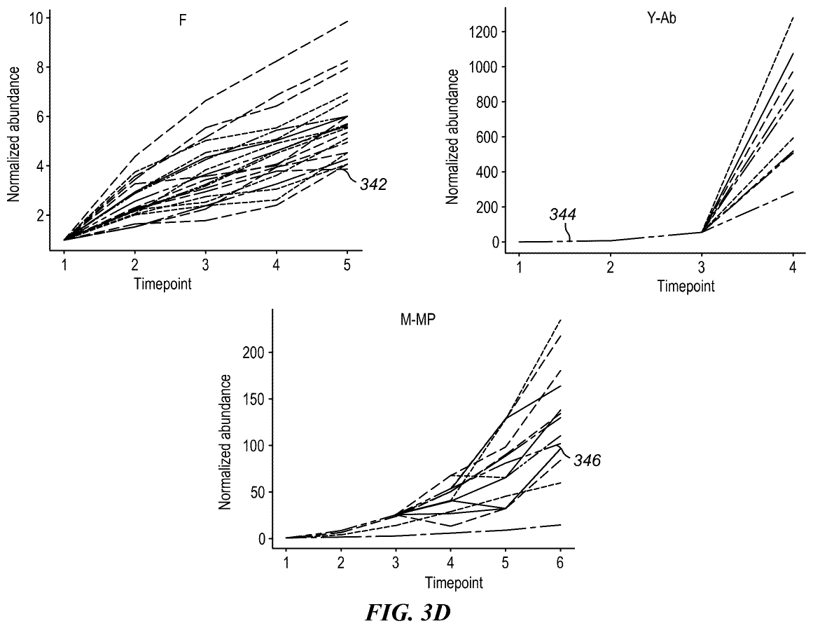

This disclosure investigated evidence for rising stars by comparing EVFI inferred fitness to final timepoint frequencies. FIG. 3A illustrates example variant frequency trajectories on datasets A, B, C, and D. FIG. 3B illustrates example variant frequency trajectories on datasets F, Y-Ab, and M-MP. FIG. 3C illustrates example normalized abundance trajectories on datasets A, B, C, and D. FIG. 3D illustrates example normalized abundance trajectories on datasets F, Y-Ab, and M-MP. Lines 302, 304, 306, 308, 310, 312, 314, 316, 318, 320, 322, 324, 326, 328, 330, 332, 334, 336, 338, 340, 342, 344, and 346 show variants with highest frequency in a timepoint. The remaining lines in FIGS. 3A-3D show rising stars. Normalized abundance trajectories are plotted by computationally adjusting all initial variant frequencies to be equal. In all seven datasets, dozens to thousands of rising stars are found with evidence of having higher fitness than the baseline, yet much lower frequency

TABLE 4
Statistics of rising star variants, with higher fitness
than the most common variant in the final timepoint.

| | | Final timepoint | Final timepoint read |
|---|---|---|---|
| | | frequency of rising stars, | count of rising stars, |
| Dataset | Number of rising stars | mean | mean |
| A | 1,465 | 0.26% | 1,204 |
| B | 3,311 | 0.0095% | 505 |
| C | 12 | 1.4% | 1,237 |
| D | 4,476 | 0.0078% | 514 |
| F | 27 | 0.054% | 5,781 |
| Y-Ab | 13 | 0.039% | 31 |
| M-MP | 154 | 0.094% | 3,988 |

(FIGS. 3A-3D and Table 4). This disclosure called rising stars as variants with over 90% probability of having higher fitness under a Dirichlet-Multinomial model to account for noise

and fitness uncertainty from low read counts (See, Section of Methods).

Table 4. Ststistics of Rising Star Variants, with Higher Fitness than the Most Common Variant in the Final Timepoint.

Rising stars had 182× lower final frequency than the baseline variant by geometric mean across the seven datasets. For instance, in M-MP, 154 rising stars have final timepoint frequency averaging 0.094%, approximately 100× lower than the baseline at 7.6%. Rising stars averaged 3,988 sequencing reads in the final timepoint in M-MP, 31 reads in Y-Ab, and hundreds to thousands of reads in A-D and F. Visual inspection of rising star trajectories confirms observed evidence of stronger enrichment, consistent over multiple timepoints, than the baseline. Together, this evidence may suggest that rising stars cannot be explained solely by sequencing errors or noise.

Empirically in these datasets, high final timepoint frequency may be largely determined by high initial frequency and less so by fitness, which may be problematic when initial frequency is determined by factors unrelated to biological activity. Rising stars have 10-100× lower initial frequencies than the baseline variant, preventing them from overtaking the baseline within the number of selections performed. If rising stars had the same initial frequency as the baseline variant, they would have dominated the baseline in frequency (FIGS. 3A-3D).

Taken together, rising stars are common and can be abundant in seven diverse datasets ranging from phage display, yeast growth, yeast display, and mRNA display, which reflects a similar report of rising stars in continuous directed evolution platforms. These findings may suggest that sampling from the final timepoint population can be suboptimal in terms of finding the highest fitness variants and may show that fitness inference can identify rising stars with observed evidence of stronger growth under selection pressure and competition.

EVFI Discovers a Sub-Nanomolar Antibody without Using all Selections

In particular embodiments, the sequencing time-series data may comprise DNA sequencing time-series data collected from a yeast display and the first biomolecule may comprise an antibody. The molecule nomination system 190 may determine, based on the inferred fitness score for the antibody, that a binding affinity of the antibody to receptor tyrosine kinase meets a predetermined criteria for selection.

This disclosure performed a yeast display campaign on antibodies to bind to the D3b and D3c domains of fibroblast growth factor receptor 1 (FGFR1). FGFR1 is a receptor tyrosine kinase involved in cell growth, differentiation, angiogenesis, and tissue repair. FGFR1 antagonists, such as monoclonal antibodies and peptide inhibitors, are in clinical trials for their potential to improve cancer treatment outcomes by inhibiting oncogenic signaling, angiogenesis, and overcome resistance to existing therapies.

The initial library was designed around a seed with 6.6 nanomolar affinity to D3b and 24.2 nanomolar affinity to D3c. Over five selection rounds, the population was iteratively split and subjected to a range of selection conditions (See, Section of Methods), ranging from 250 nM target concentration to as low as 100 pM.

A human expert scientist sampled clones from populations after four or five rounds of selection to nominate variants for testing. In total, 71 clones were sampled yielding 25 unique variants. All expressed successfully, and surface plasmon resonance (SPR) was used to measure their binding affinities to D3b. In addition, five of the tightest D3b binders were manually chosen to measure their D3c binding affinity.

This disclosure hypothesized that EVFI, by identifying strongly enriching variants with low frequencies, could nominate tight binders earlier. The embodiments disclosed herein used Illumina DNA sequencing to read the full heavy chain and a portion of the light chain, which this disclosure collectively refers to as “truncated” variants. The embodiments disclosed herein used EVFI to infer fitness from sequencing data of the first four selection rounds, excluding data from the fifth selection round. The embodiments disclosed herein nominated truncated variants and extended them with the seed light chain to obtain full-length antibody variants for testing. Among 25 variants nominated with EVFI, 20 expressed and 19 bounded.

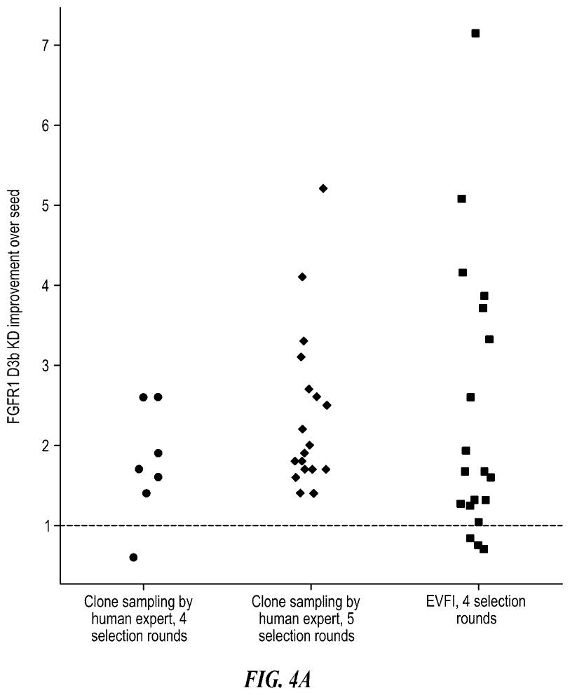

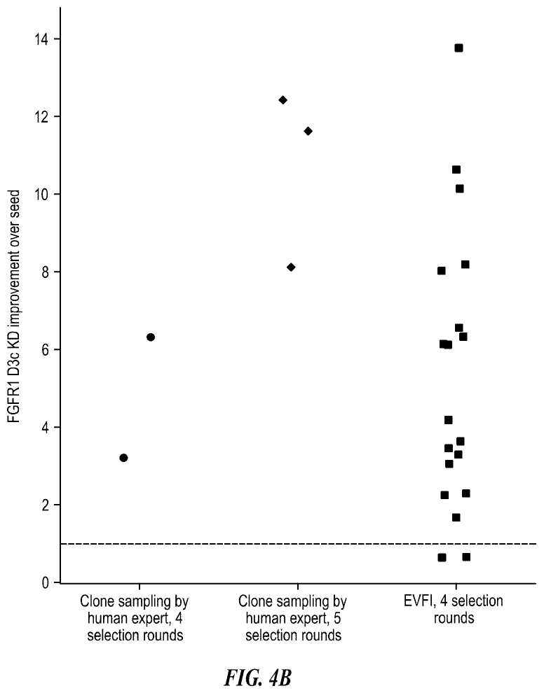

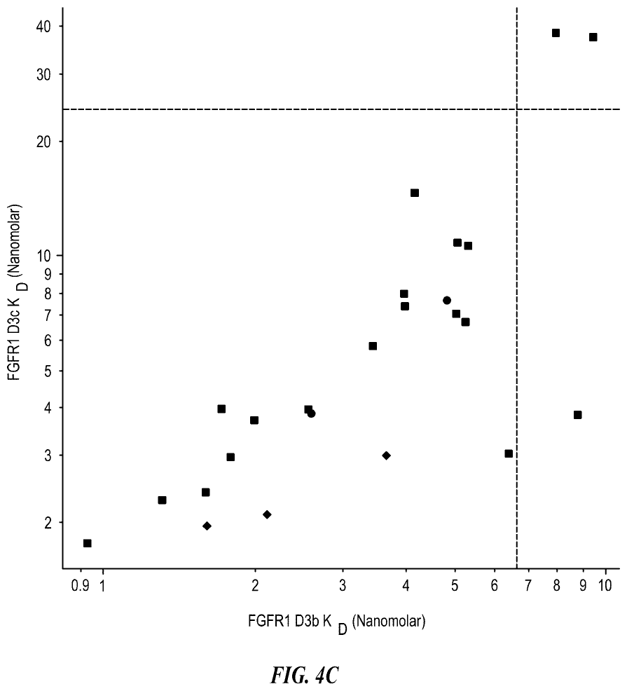

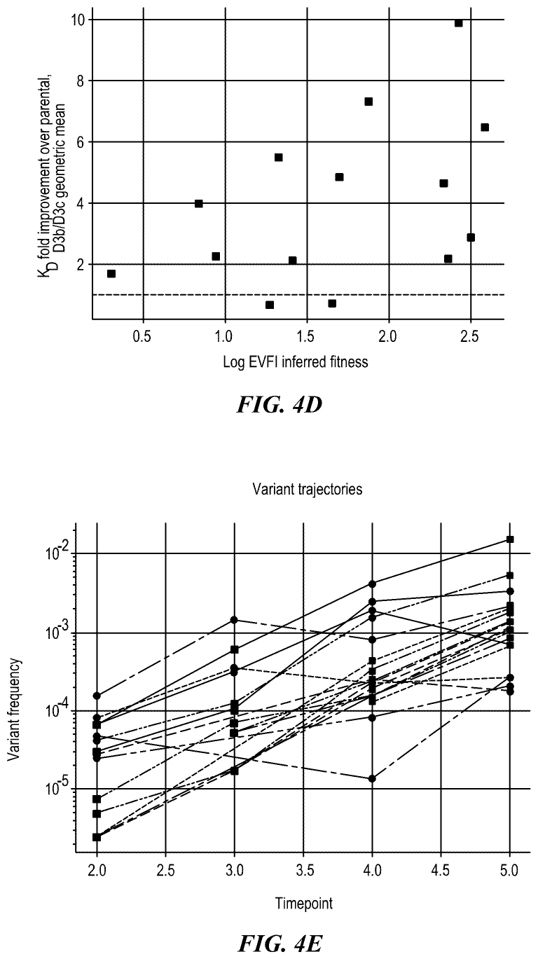

The embodiments disclosed herein compared binding affinities to D3b and D3c of three groups of variants: human-expert-nominated variants after four selection rounds, which this disclosure denotes as the “H-4S” group; the same after five selection rounds (“H-5S”), and EVFI-nominated variants after four selection rounds (“EVFI-4S”). FIG. 4A illustrates an example scatterplot of K_{D} for D3b. FIG. 4B illustrates an example scatterplot of K_{D} for D3c. FIG. 4C illustrates an example scatterplot of K_{D} for both D3b and D3c. K_{D} measured by surface plasmon resonance (SPR). Square points depict variants nominated by EVFI without using data from the final timepoint, and round depicts human expert nominations by sampling clones from the final timepoint. Dashed line with indicate seed sequence. FIG. 4D illustrates an example comparison of log EVFI inferred fitness of nominated truncated variants and the geometric mean of K_{D} fold improvement on binding D3b and D3c over seed of full-length nominated variants. Spearman ρ=0.51, p=0.06, N=14. FIG. 4E illustrates example frequency trajectories of variants. In all panels, square dots and associated lines depict EVFI-nominated variants using four selection rounds, round dots and associated lines depict human expert nominations by sampling clones from the final.

Overall, the EVFI-4S group contained substantially tighter binders than H-4S and found comparable binders to H-5S. For D3b affinity, none (0/7) of the H-4S group improved seed affinity by more than 3×, while 32% (6/19) of EVFI-4S did, a similar rate to H-5S at 22% (4/18) (FIG. 4A). For D3c affinity, no H-4S variants improved seed affinity by more than 10×, while two H-5S variants did and three EVFI-4S did (FIG. 4B). Many EVFI-4S variants (37%; 7/19) had equal or tighter affinity to D3b and D3c than the best H-4S binder.

The best dual-binding variant in any group was in EVFI-4S which improved D3b affinity 7.1× to 930 pM and D3c affinity by 13.8× to 1.76 nM (FIG. 4C). Notably, EVFI-4S variants were selected on with target concentration at 1 nM or greater, while H-5S variants were selected on at target concentrations down to 100 pM. Collectively, these results may suggest that EVFI discovers tighter binders than clone sampling given the same rounds of selection, and EVFI can discover comparable binders earlier than clone sampling performed with an additional round of selection at higher selection stringency.

Among EVFI-nominated variants, inferred fitness scores were positively correlated with combined D3b and D3c affinity improvement over seed (ρ=0.51, p=0.06, N=14; Spearman correlation) (FIG. 4C). EVFI-4S demonstrate strong and consistent enrichment over multiple timepoints compared to H-4S variants, which have inconsistent and weaker enrichment trajectories. EVFI-4S variants had an average frequency of 0.25% after four selection rounds.

Overall, these results demonstrate that EVFI variant nomination can perform similarly, and in some cases exceed, sampling from the population after performing an additional selection round, even at weaker selection stringencies. EVFI may thus enable identifying strong variants earlier with reduced experimental effort.

### DeepEVFI Improves Macrocyclic Peptide Affinity

In particular embodiments, the sequencing time-series data may comprise DNA sequencing time-series data collected from an mRNA display and the first biomolecule may comprise a macrocyclic peptide. The molecule nominations system may determine, based on the inferred fitness score for the macrocyclic peptide, that a binding affinity of the macrocyclic peptide to one or more domains of receptor tyrosine kinase meets a predetermined criteria for selection.

This disclosure performed two mRNA display campaigns on macrocyclic peptides to bind to the YAP binding domain of TEAD2. TEAD proteins are transcription factors that regulate the downstream effects of the Hippo signaling pathway that controls organ size and development. TEAD serves as a receptor for its co-activators YAP/TAZ to upregulate cell proliferation and survival. Enhanced activity of YAP/TAZ and TEAD have been observed in various human cancers. Macrocyclic peptides have proven highly effective for engaging previously deemed “undruggable” shallow grooves and clefts observed in many protein-protein interactions such as TEAD-YAP.

In the first campaign, a naive randomized library was used in mRNA display. The final timepoint of campaign 1 was Sanger sequenced and 21 variants were nominated by a human expert using frequency ranking. Twenty of these variants were re-nominated with GKK tags added to them to improve solubility, resulting in 41 total variants. Twelve of these 41 variants had binding activity and K_{D} successfully determined using SPR, with affinity ranging from 1-100 micromolar. A second campaign was designed based on the first campaign, and seven rounds of mRNA display were performed. Based on the results in the first campaign, all following tested variants had GKK tags added.

A human expert manually nominated six variants using frequency ranking from Illumina sequencing data of the final timepoint population, while also aiming to nominate diverse variants. Out of six variants, two did not bind, two had measurable binding affinity but their K_{D} could not be determined, and two were binders with K_{D} of 380 nM and 500 nM.

Particular embodiments applied DeepEVFI on the DNA sequencing data of the second campaign by jointly inferring fitness and learning a sequence-to-fitness deep neural network. Particular embodiments nominated a set of variants using deep inferred fitness, while also designing for variant diversity (See, Section of Methods), without knowledge of the six human-expert nominated variants, which yielded one duplicate renomination of the 380 nM binder.

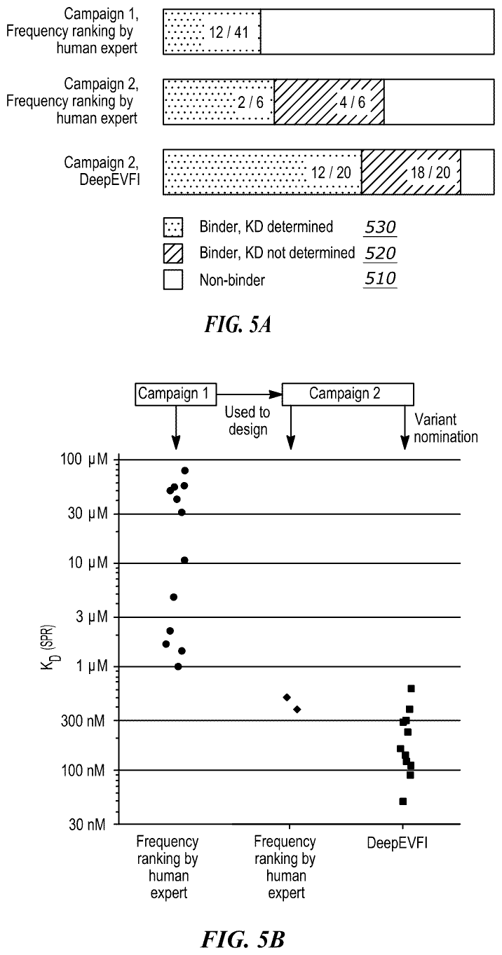

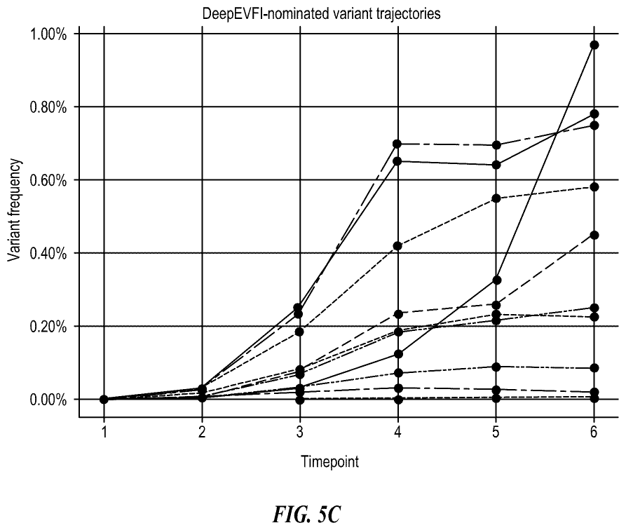

FIG. 5A illustrates an example diagram depicting SPR outcomes for nominated variant sets on macrocyclic peptides binding to TEAD. The outcome categories include non-binding 510, binding but K_{D} is undetermined 520 due to curve fit quality, and binding with K_{D} determined 530. FIG. 5B illustrates example TEAD K_{D} for variants. FIG. 5C illustrates example trajectories of DeepEVFI-nominated variants in M-MP dataset. Among DeepEVFI-nominated variants, 90% (18/20) bound, and affinity was determined for 12 variants (FIG. 5A). Nearly all DeepEVFI variants (92%; 11/12) matched or improved binding affinity over the best human-expert nominated variant, which DeepEVFI also renominated. The single best discovered binder had 50 nM affinity, which is 7.6× tighter than the best human-nominated variant from campaign 2, and 20× tighter than the best from campaign 1 (FIG. 5B). Notably, DeepEVFI-nominated variants had an average frequency of 0.2% in the final timepoint (FIG. 5C), and the tightest binder had a frequency of 0.02% in the final timepoint. The discovery of 8-mer macrocycle with 50 nM affinity to TEAD approaches the 15 nM affinity of a previously reported 17-mer macrocycle, despite being shorter.

Overall, these results demonstrate that DeepEVFI using deep DNA sequencing of all timepoints from a directed evolution campaign can nominate variants with tighter binding affinity that are overlooked by common variant nomination strategies like frequency ranking.

As shown by the discoveries above, another technical advantage of the embodiments may include more effective selection of discovered hits as the molecule discover system infers fitness of biomolecules that indicates biological activity and then determines discovered hits based on such biological activity.

Variant Nomination with Diversity

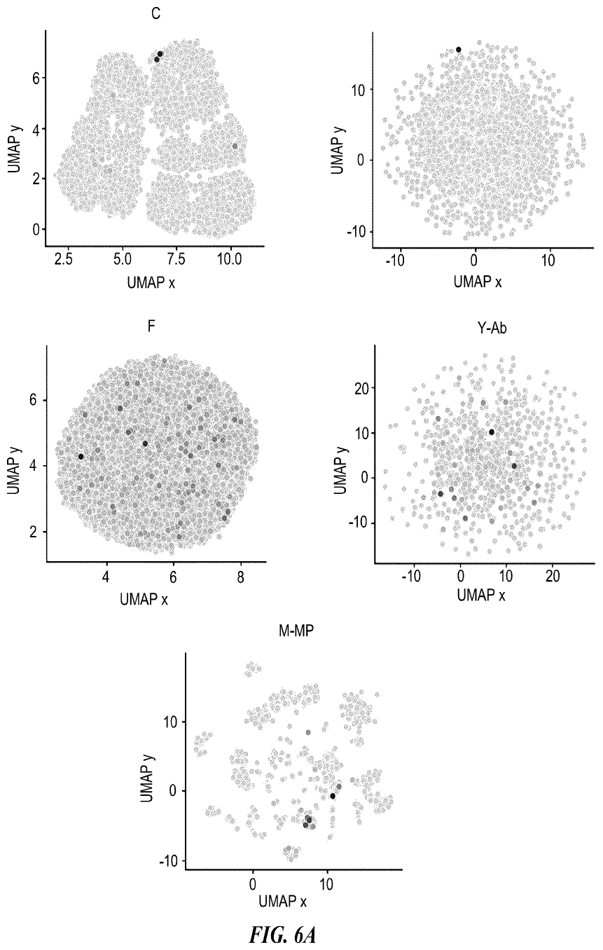

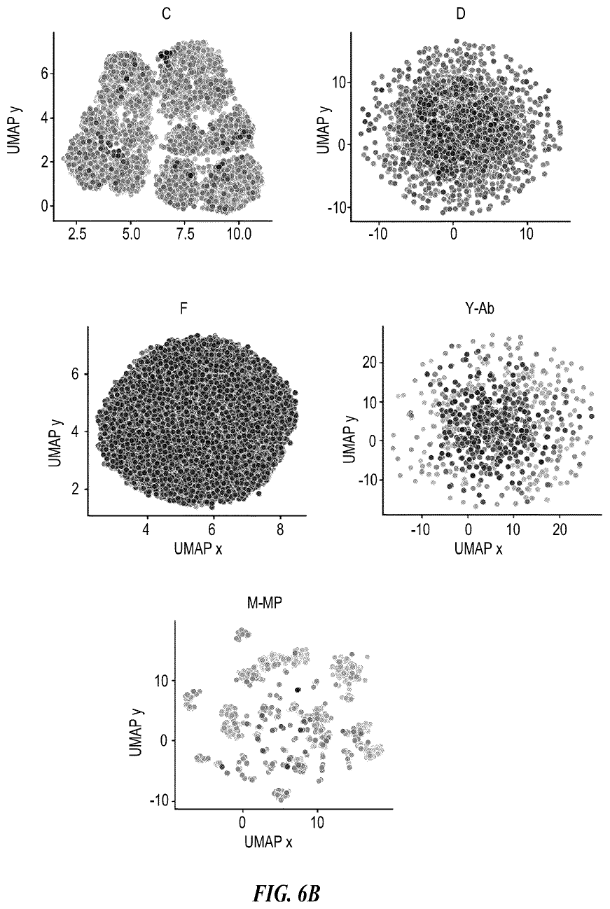

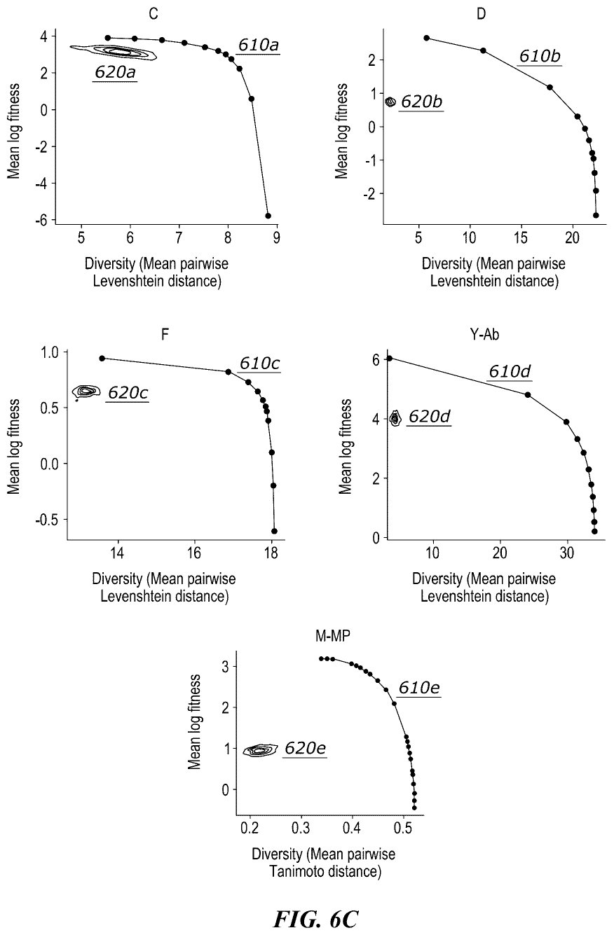

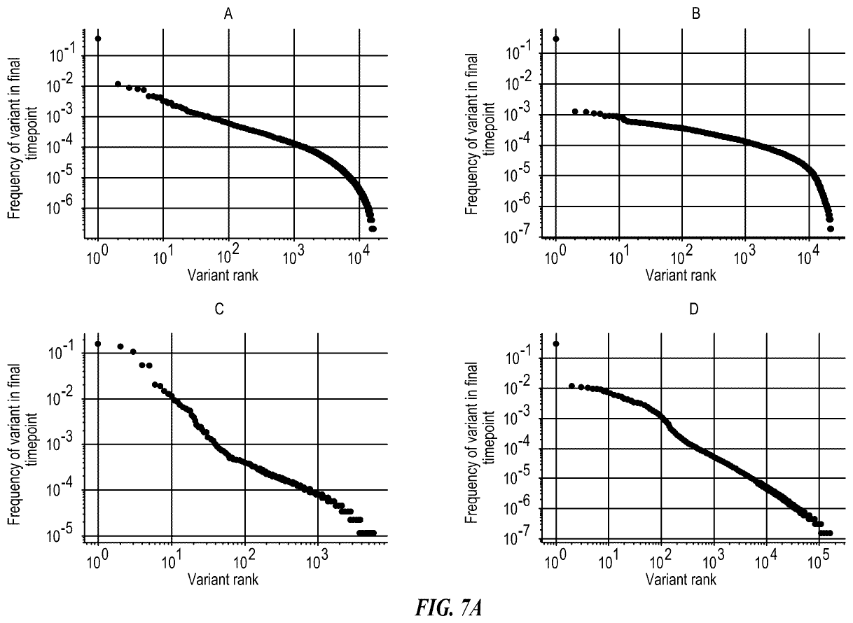

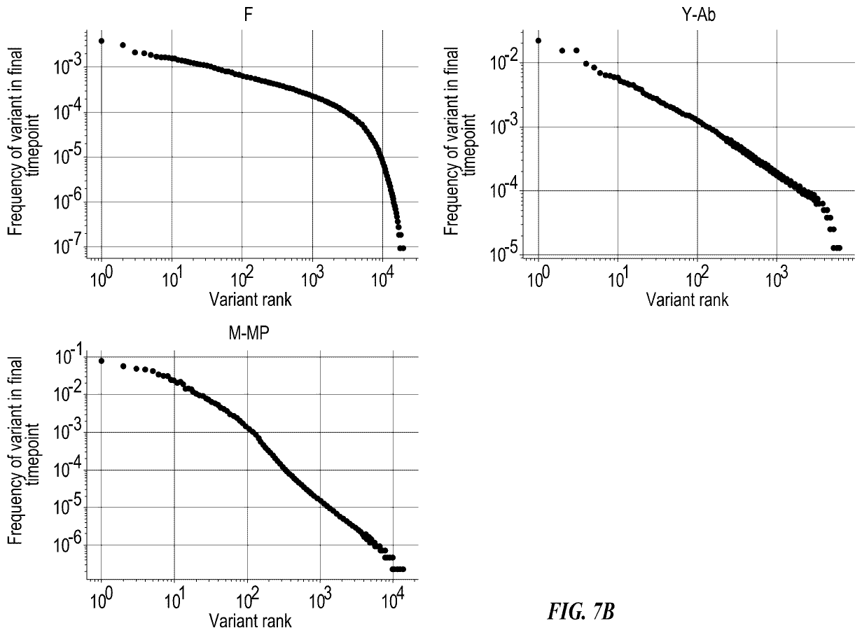

FIG. 6A illustrates example UMAP plots of sequences. The shade intensity reflects final timepoint frequency. FIG. 6B illustrates example UMAP plots of sequences. The shade intensity reflects log inferred fitness. FIG. 6C illustrates example Pareto frontier of nomination sets with 96 variants optimizing average log fitness or average pairwise distance 610 compared to nomination sets by sampling 96 variants by final timepoint frequency 620. FIGS. 7A-7B illustrate example sorted frequencies of variants in final timepoints.

Discovering diverse hits can be important for lead discovery in drug development. Diversity is a priority when attempting to solve multi-objective optimization problems without full knowledge of all the objectives or the ability to measure all properties related to objectives. Despite campaigns undergoing two to seven rounds of selection in the seven datasets studied herein, none of the populations converged to a small handful of dominant variants, and instead had high diversity with thousands to tens of thousands of unique variants, with frequencies generally following an exponential distribution (FIGS. 7A-7B). Visual inspection of UMAP plots reveal that high final timepoint frequency covers little sequence diversity, while variants with high inferred log fitness span much more of the sequence diversity in the population (FIGS. 6B-6C). This disclosure hypothesized that fitness inference could be used to nominate diverse variant sets with high inferred fitness, reflecting evidence of consistent strong enrichment over timepoints, which may better represent the large sequence diversity present in evolved populations.

Across the five datasets with genotype information, final timepoint sampling produces variant sets with substantially lower diversity and lower fitness than variant sets built using full time-series DNA sequencing data (FIG. 6A). Particular embodiments build variant sets with varying trade-offs between high inferred log fitness and high sequence diversity using a greedy algorithm (See, Section of Methods), which in all cases yields variant sets with higher mean fitness, higher diversity, or both, compared to final timepoint sampling. In datasets D and Y-Ab, final timepoint sampling nominates variants sets with less than five-point mutation difference between variant pairs on average, while particular embodiments build nomination sets that differ by more than 20 mutations from each other on average in dataset D, and more than 30 mutations in Y-Ab. In datasets Y-Ab and M-MP, the top fitness-optimized variant sets have 7.4× higher fitness than variant sets from final timepoint sampling. Taken together, fitness inference can also support nominating diverse variants that can better capture the sequence diversity in evolved populations.

As can be seen, another technical advantage of the embodiments may include the ability to discover novel and diverse variants as the molecule nomination system 190 may identify variants with high fitness but low frequency, which may be missed through standard approaches for biomolecule discovery.

### Discussion

In computational evaluations, this disclosure showed that EVFI and DeepEVFI outperform ACIDES and Enrich2 at predicting enrichment in held-out selection rounds in seven real-world datasets including yeast, phage, and mRNA display. While all model enrichment in iterated selections, this performance difference may be due to EVFI and DeepEVFI's intentional design in supporting directed evolution with potential mutations, whereas Enrich2 is primarily designed for gene enrichment analysis with two timepoints, and ACIDES assumes that mutations do not introduce new variants. In the disclosed investigations, Enrich2 fails to properly account that linear dynamics across two timepoints imply nonlinear dynamics over three or more timepoints, and ACIDES' no-mutation assumption causes overly optimistic scoring of variants absent in early timepoints. This disclosure sheds light on the potential for fitness inference methods to improve variant nomination by finding that rising stars, which may be missed by conventional nomination methods, are common in a variety of datasets.

In yeast display and mRNA display, on antibodies and macrocyclic peptides, that EVFI and DeepEVFI enable finding tight binders with low frequency that may easily be missed by conventional nomination approaches like clonal sampling and final timepoint frequency ranking. Beyond hit discovery, by finding diverse variants with comparable or improved affinity compared to human expert picks, EVFI and DeepEVFI supports hit expansion which may assist lead discovery. Large sequence-fitness datasets may shed light on sequence-activity relationships, assist researchers in future designs and follow-up studies such as testing mutation reversions, and provide alternative leads for development towards the clinic.

Experimental conditions may impact fitness score quality: if enrichment is caused more by nuisance factors than by the primary activity of interest such as binding affinity, EVFI or DeepEVFI may be less useful for nominating improved variants. EVFI may thus benefit from careful design of selection conditions, but EVFI can also be used as a framework for quantifying selection design quality, by considering the correlation between denoised enrichment over multiple timepoints with measurements of the primary activity of interest. This quantification may provide a path for improving selection design.

### Methods

### Yeast Display on FGFR1

The CDR-H3 from clone 1C2 was subsequently incorporated into anew scFv YSD library with a diversity of 2×10⁸, introducing non-germline encoded diversity in CDR-L1, CDR-L3, CDR-H1, and CDR-H2. The library was adapted into pCTCON2 and transformed by electroporation. This library formed a starting population called A in this disclosure. Selection was performed iteratively with splitting some populations and subjecting each split to different selection conditions, to yield a total of seven final populations, with a total of 15 intermediate populations A-O. The seven tracks yielding each final population is: 1) ABCDE, 2) ABCDFJ, 3) ABCDFK, 4) ABCDGL, 5) ABCDHM, 6) ABCDHN, 7) ABCDIO. The selection conditions yielding each population is B: MACS against 125 nM D3b and 125 nM D3b in 500 microliter beads; C: FACS on 10 nM D3b and 10 nM D3c; D, E: 10 nM D3b/D3c; F: 1 nM D3b/D3c; G/L: 100 pM D3c; H: 1 nM D3b/D3c with 1-hr FACS buffer wash; I/O: 1 nM D3c with 1-hr FACS buffer wash with 1 μM untagged competition; J: 1 nM D3b; K: 1 nM D3c; M: 1 nM D3b with 1-hr FACS buffer wash; N: 1 nM D3c with 1-hr FACS buffer wash. Clones were sampled from all final populations, which were E with four prior selection rounds, and J, K, L, M, N, and O with five prior selection rounds. Sampled clones were reformatted into human IgG1 and analyzed by SPR. Timepoint populations after each selection were also subjected to next-generation sequencing (NGS) using the Illumina 2×300 MiSeq platform. Given the 600 base pair limits of this method, each output was amplified from the Light Chain Framework 3 through the entire Heavy Chain. Sequencing data were then analyzed using EVFI and DeepEVFI as described in this disclosure. Selected designs from this analysis were expressed, and SPR was performed.

### FGFR1 Naive Yeast Display Library Construction & Seed Antibody Isolation

A single-chain variable fragment (scFv) library was designed in a VL-linker-VH format and synthesized. The design incorporated diversity predominantly within the CDR-H3 region while the remaining CDR's (Complementary Determining Regions) were germline encoded. The library utilized germline scaffolds heavily prevalent in the human repertoire and previous clinical antibodies. Sequences coding for the overlap region with the pCTCON2 vector were appended as appropriate to the scFv design allowing for homologous recombination in yeast. The pCTCON2 vector was prepared for homologous recombination by digestion overnight with SalI-HF followed by digestion overnight with NheI-HF and BamIHF. The library was amplified via PCR using Q5 HotStart High-Fidelity Polymerase. The amplified scFv library insert and the digested vector were transformed into RJY100 yeast strain, using previously described electroporation methods. The final library contained 1.6×10⁹ members.

Sorts for FGFR1-D3b & FGFR1-D3c binders were conducted using previously described yeast surface display techniques. Initial rounds of sorting were conducted using Magnetic Activated Cell Sorting (MACS), ensuring at least tenfold coverage of the library or population size in each round. After two rounds, three subsequent sorting rounds were completed by Fluorescent Activated Cell Sorting (FACS). Each round, biotinylated FGFR1-D3b and/or biotinylated FGFR1-D3c was used for selection at decreasing concentration. During MACS rounds, Streptavidin Microbeads were coated with biotinylated selection agent prior to mixing with the scFv library. For FACS rounds, the biotinylated selection agent acted as the primary and streptavidin AlexaFluor 488 conjugate (1:500) as secondary. Successful scFv display was assessed using chicken anti-cMyc primary (1:500).

Following MACS and FACS selections, single yeast colonies were plated on SDCAA agar plates, and 96 individual clones were sequenced by Sanger sequencing. Unique clones were reformatted into human IgG1 antibodies using DNA synthesis. The resulting IgG1 constructs were transiently expressed in CHOK1 cells (30 mL cultures) and purified using previously described expression and purification methods. The IgG1 antibodies were evaluated using Surface Plasmon Resonance (SPR) on a bioanalytical sensing machine. The machine was primed with HBS-EP buffer (0.01 M HEPES pH 7.4, 0.15 M NaCl, 3 mM EDTA, 0.005% v/v Surfactant P20), and antigens or antibodies were also diluted into the same buffer for all SPR experiments. Antibodies were captured by Protein A at 2 μg/mL, and Multi-Cycle Kinetics was performed at 25° C. to determine binding affinities to both FGFR1-D3b and FGFR1-D3c. All data were analyzed using a 1:1 binding model. A single clone, designated 1C2, was selected for affinity maturation, demonstrating binding affinities of 6.6 nM to FGFR1-D3b and 24.2 nM to FGFR1-D3c.

mRNA Display on TEAD

The embodiments disclosed herein designed an eight amino acid macrocyclic lariat library in a genetically reprogrammed in vitro translation system as previously described. Briefly, particular embodiments aminoacylated ClAc-L-phenylalanine onto the initiator tRNA^{fMet} and encoded cys-teine onto the codon CCA at positions 5, 6, 7 or 8 to spontaneously from a thioether link with the cyclic portion of the peptide ranging from a ring size of 4-8. All peptide sequences were followed by a sequence encoding for a G3SGS linker. The remaining positions were randomized with a NNU genetic code containing N-methyl-L-phenylalanine (MeF; codon TTC), D-valine (v; codon ATC), tert-Butyl-glycine (tBuG, codon CCT), 4-Phenyl-L-phenylalanine (Bip; codon ACC), (S)-1,2,3,4-Tetrahydroisoquinoline-3-carboxylic acid (Tic; codon GCC), L-4-thiazolyl-Alanine (4TzA, codon GAT), (4-hydroxybenzyl)glycine (HyG, codon TGT) in addition to the eight natural amino acids leucine, valine, serine, glycine, histidine, arginine, asparagine, tyrosine.

Affinity selections of macrocycles binding to TEAD were conducted using biotinylated N-terminal avi-tagged TEAD2-YBD, expressed and purified as described previously. For affinity selections each translation reaction contained 2 μM mRNA-peptide-linker conjugate, 50 μM of each initiator tRNA and 25 μM of each elongator tRNA. Translation reactions were quenched by addition of 17 mM EDTA and reverse transcribed using RNase H minus reverse transcriptase at 42° C. for 30 minutes. Following a buffer exchange into HBS-T buffer (25 mM HEPES-NaOH pH 7.5, 150 mM NaCl, 0.05% Tween-20), the macrocycle:cDNA library was incubated with 250 nM biotinylated TEAD2-YBD for 20 minutes at room temperature and then incubated with streptavidin-coated beads (Dynabeads M-280 Streptavidin) for 10 minutes. The beads were washed three times with HBS-T. The cDNA was eluted by heating the beads for 5 min at 95° C. and the recovery of the elution fraction was determined by qPCR using SYBR Green I on a thermal cycler. The enrichment of peptides for each round was assessed by next-generation sequencing using a HiSeq sequencer of the final cDNA after each round.

### SPR for Macrocyclic Peptides

SPR experiments for macrocyclic peptides were performed in HBS-T (150 mM NaCl, 50 mM HEPES pH 7.5, 0.001% Tween 20, 0.2% PEG3350) in 2% DMSO. Peptides were diluted six times in a threefold dilution series from 1 mM and injected at 50 mL/min using single-cycle kinetics with 120 s association and 1500 s dissociation. The SPR data was evaluated for binding affinity.

### Evolutionary Fitness Inference (EVFI)

Evolutionary fitness inference (EVFI) may use input data in the form of a count table C with shape G×T, with G unique genotype variants, and T total timepoints, and entries in the count table may be non-negative integers representing DNA sequencing counts. The main output may include an inferred G-dimensional fitness vector w, where each variant's fitness may be a positive number representing its growth rate over multiple rounds of selection and competition.

In EVFI, particular embodiments focus on inferring relative fitness w without explicitly inferring τ and η to improve efficiency, while handling their potential impact on estimating w to retain accuracy.

There may be two properties of the dynamics in equation (4), under the situation where there is no mutation, and all variants are present at all timepoints. First, if there is no measurement noise (i.e., pₜ is observed), then w may be identifiable given data from just two timepoints under equation (4). Second, if a variant has zero frequency at time t, then that variant may have zero frequency at all following timesteps.

When mutations can introduce variants into populations that follow the dynamics of equation (4), the first property may be modified: if there is no measurement noise, then w may be identifiable given data from just two timepoints under equation (4) for all variants that are present in the earlier timepoint. However, variants that are absent, and enter the population at a later timepoint, may not have their fitness identified.

The embodiments disclosed herein exploit these properties to propose a masked optimization procedure, where particular embodiments may iterate over consecutive timepoint pairs t,t+1, and optimize the estimate of w for variants with non-zero count at timepoint t. This disclosure denotes maskₜ(⋅) as a function that selects the elements in an input G-dimensional vector at indices where c_{g,t}>0. The python pseudocode for masking may be:

```
def mask(input, t):
    counts_t = get_counts(t)
    # input has length G, counts_t has length G
```

return [input[i] for i in range(len(input)) if counts t[i]>0]

Particular embodiments may randomly initialize w and optimize w using maximum likelihood for a likelihood function [symbol] , which can be a Multinomial distribution in this disclosure:

$${max}_{w} {\prod }_{t = 1}^{T - 1} ℓ ( {mask}_{t} ( {C}_{t + 1} ) ; p = {mask}_{t} ( \frac{w}{{w}^{T} {C}_{t}} ⊙ {C}_{t} ) , N = {\sum }_{g} {c}_{g , t + 1} ) \qquad ( 6 )$$

$$where$$

$${\hat{p}}_{t + 1} {=}^{△} \frac{w}{{w}^{T} {C}_{t}} ⊙ {C}_{t} \qquad ( 7 )$$

simulates forward in time equation (4) using current estimate of relative fitness w and observed variant counts Cₜ. Note that \hat{p}ₜ₊₁ may be guaranteed to be a probability vector that sums to one, and the equation may be equivalent if one replaces Cₜ with the observed variant frequency vector with elements

$${c}_{g , t} / {\sum }_{g}^{r} {c}_{g , t} .$$

The symbol ⊙ indicates element-wise multiplication of two vectors.

Particular embodiments may optimize this loss using batched gradient descent, batching over time but not over genotype variants. Each batch may correspond to one pair of timepoints t,t+1 and all genotype variants.

In summary, EVFI may sidestep explicitly modeling when variants enter the population and their initial frequencies to focus on inferring relative fitness, by using masking during optimization to focus on fitness inference using variants that are present in the population. In particular embodiments, the first biomolecule may be within the population of biomolecules in the plurality of rounds and present in a second-to-last round of the plurality of rounds. The molecule nomination system 190 may predict, based on the inferred fitness score for the first biomolecule, a biomolecule frequency of the first biomolecule in a last round of the plurality of rounds.

### DeepEVFI

In particular embodiments, the molecule nomination system 190 may access a biomolecule representation of the first biomolecule and process the biomolecule representation of the first biomolecule by a machine-learning model (e.g., DeepEVFI). The molecule nominations system may then output the inferred fitness score for the first biomolecule by the machine-learning model based on the processing of the biomolecule representation of the first biomolecule.

For DeepEVFI, one can use a different approach which jointly infers relative fitness function w(g; θ) parametrized by θ, and initial abundances η. DeepEVFI may jointly learn a sequence-to-fitness deep neural network parametrized by θ, and initial abundances of each variant. DeepEVFI may first empirically compute a vector τ where each element is the earliest timepoint where each variant has non-zero count. The predicted abundance of a variant g at any time t≥τ_{g} may be:

$${\hat{n}}_{g , t} = \{ \begin{matrix}0 , t < {τ}_{g} \\ {n}_{g} t , = {τ}_{g} \\ {w ( g ; θ )}^{( t - {τ}_{g} )} {n}_{g ,} t > {τ}_{g}\end{matrix} \qquad ( 8 )$$

Particular embodiments may use predicted abundances to predict frequencies:

$${\hat{p}}_{g , t} {=}^{△} \frac{{\hat{n}}_{g , t}}{{\sum }_{g}^{′} {\hat{n}}_{{g}^{′} , t}} \qquad ( 9 )$$

As described above, training the machine-learning model (e.g., DeepEVFI) may comprise identifying, based on the sequencing time-series data, one or more first biomolecule frequencies associated with one or more first biomolecules of the population of biomolecules that are greater than zero in a first round of the plurality of rounds. The training may then comprise identifying, based on the sequencing time-series data, one or more second biomolecule frequencies associated with the one or more first biomolecules in a second round consecutive to the first round. The training may additionally comprise predicting biomolecule frequencies associated with the one or more first biomolecules in the second round given the one or more first biomolecule frequencies. The training may also comprise calculating a loss between the predicted biomolecule frequencies and the second biomolecule frequencies. The training may further comprise determining baseline fitness scores for the population of biomolecules based on the loss. In particular embodiments, learning the inferred fitness scores for the population of biomolecules may be based on the baseline fitness scores for the population of biomolecules.

Particular embodiments may use a Dirichlet-Multinomial maximum likelihood alongside \hat{α} (a concentration parameter for the Dirichlet-Multinomial distribution) to learn θ and η:

$$ℓ ( θ , η , \hat{α} ) {=}^{△} \begin{matrix}max \\ θ , η , \hat{α}\end{matrix} {\prod }_{t = 1}^{T} DirichletMultinomial ( {C}_{t + 1} ; α = \hat{α} {\hat{p}}_{t + 1} ) \qquad ( 10 )$$

In other words, learning the inferred fitness scores in the training of the machine-learning model may comprise optimizing a Dirichlet-multinomial loss function. In particular embodiments, the training of the machine-learning model (e.g., DeepEVFI) may further comprise calculating a Dirichlet loss negative log-likelihood between the predicted biomolecule frequencies and actual biomolecule frequencies as a negative log-likelihood.

Particular embodiments may optimize this loss using batched gradient descent, batching over time but not over genotype variants. Each batch may correspond to one pair of timepoints t,t+1 and all genotype variants. Mini batching over genotype variants as an implementation of minibatch stochastic gradient descent may appear to work here because dynamics equation (4) holds for subsets of genotype variants. However, due to that equation (4)'s invariance to rescaling relative fitness (See, Property 2), each minibatch may learn a different, arbitrary fitness scale which is not directly comparable to the fitness scale of other minibatches. This can substantially hinder training efficiency and the ability to find a good optimum in a reasonable amount of time.

Particular embodiments may use a three-stage approach to training DeepEVFI. First, particular embodiments may train EVFI on the input count table to obtain a fitness vector w. Second, particular embodiments may warm-up train the deep neural network with mean-squared-error loss on predicting w. Third, particular embodiments may perform full end-to-end training combining count table likelihood loss with mean-squared-error loss. Denoting the neural network as f_{θ}:X→ [symbol] , the loss in the third stage is:

$${c}_{0} ℓ ( θ , η , \hat{α} ) + {c}_{1} {\sum }_{i} {( {f}_{θ} ( {x}_{i} ) - {w}_{i} )}^{2} \qquad ( 11 )$$

where c₀, c₁ are weights that may vary during training. Particular embodiments can avoid optimization issues caused by the non-identifiability property by not mini batching over variants. The three-stage approach including obtaining a fitness vector, training the deep neural network with mean-squared-error loss on predicting the fitness vector, and performing full end-to-end training, combining count table likelihood loss with mean-squared-error loss may be an effective solution for addressing the technical challenge of training efficiency and the ability to find a good optimum in a reasonable amount of time as such approach can avoid optimization issues caused by the non-identifiability property by not mini batching over variants.

In particular embodiments, the machine-learning model (e.g., DeepEVFI) was trained using sequencing time-series data associated with biomolecule frequencies of particular biomolecules. The sequencing time-series data was obtained from directed evolution of a population of biomolecules over a plurality of rounds. The population of biomolecules in each round was a unique set of biomolecules with respect to each other round.

### Remarks

This section remarks on the problem framework, the design of EVFI and DeepEVFI, and their properties.

EVFI may support parallel tracks and directed acyclic graph timepoint relationships. This is a property of EVFI, but not DeepEVFI, because EVFI's loss function can be decomposable into terms depending only on input/output timepoint pairs (t,t+1 conventionally), and EVFI may not explicitly model frequencies unlike DeepEVFI and other methods.

Consider a data structure representation of time series data where each node corresponds to a timepoint, and directed edges connect input timepoints to output timepoints for each round of directed evolution. The simplest type of dataset may be where a node follows nodes in a single “line” such as o→o→o. However, in practice, multiple directed evolution campaigns might be performed on different populations on the same target, yielding parallel tracks: o→o→o; o→o→o. Alternatively, a population might be split and subjected to two similar new rounds of selection, yielding a bifurcation and a tree structure: o←o→o.

Methods that explicitly model frequencies may need to learn many sets of initial frequencies on more complex datasets. The total parameter count that is optimized thus can increase with increasingly complex datasets. For instance, parameter count would scale linearly in the number of parallel tracks in a dataset. In contrast, EVFI has a constant number of inference parameters regardless of dataset complexity, because its loss function can be decomposed onto edges in timepoint relationship graph. Using a loss function that can be decomposed onto edges in timepoint relationship graph may be an effective solution for addressing the technical challenge of increasing parameters to be optimized with increasingly complex datasets as there is a constant number of inference parameters regardless of dataset complexity.

Dirichlet-Multinomial distribution may model a form of genetic drift. The Dirichlet-Multinomial distribution can be an over dispersed multinomial distribution and may be equivalent to a hierarchical model where a probability vector p is drawn from a Dirichlet distribution with parameter α, then an observation is drawn from a multinomial distribution with probability vector p and number of trials N. In the context of evolutionary fitness inference, using a Dirichlet-Multinomial observation distribution can be understood as modeling a form of genetic drift, where there is random variation around each variant's growth from the previous timepoint. Using the Dirichlet-multinomial loss to optimize the fitness inference task may be an effective solution for addressing the technical challenge of the noise and fitness uncertainty from low read counts as the Dirichlet-multinomial loss may model a form of genetic drift, where there is random variation around each variant's growth from the previous timepoint.

Although the method disclosed herein may be general, the method design is partially motivated by several empirical properties of directed evolution assays such as mRNA display, yeast display, and phage display, which in turn may potentially limit the method's performance on datasets with significantly different properties. Display assays empirically may support very large population sizes of hundreds of millions to trillions of variant units that are acted upon by selection pressure—denote this number N. At the same time, mutation rates may be relatively low, up to 10⁻⁵ mutations per nucleotide per replication at the highest, denoted as μ. The disclosed simple model of variants entering the population, potentially due to mutation, is motivated by the empirical property that μ<<1, so that in practice, new mutants enter the population at very low frequency, and mutation does not meaningfully reduce the abundance of the parental variant, allowing for ignoring its effect on the parental variant abundance. If the mutation rate was much higher, then explicitly modeling the effect of mutation on the parental variant abundance would likely become necessary. Large population sizes N can reduce the importance of explicitly modeling genetic drift.

The disclosed framework assumes that variants enter the population up to one time. With explicit sources of mutation, this assumption can easily be violated in practice; however, this disclosure argues here that this violation may be not restrictive and make little difference for fitness inference. In property 4, this disclosure proves that in the disclosed framework, frequency-weighted fitness increases monotonically over time. In property 5, this disclosure proves in disclosed framework, v-trajectories are not possible, which is when a variant decreases in frequency then increases in frequency. Taken together, one can see that even if a variant truly does enter the population a second time, if the population follows the assumed dynamics, then the variant may have low fitness, because it previously entered the population, but was outcompeted. When the variant enters the population a second time, the population may have only become more competitive (its frequency-weighted fitness is now higher), so the variant will continue to be outcompeted. While accurate fitness inference for such a variant can be challenging when its frequencies are so low, the disclosed methods may generally infer low fitness for such variants.

The disclosed framework assumes that fitness does not vary by time. A simple way to account for time-varying selection pressure in this framework may be to artificially add more “time” or “generations” between timepoints that have relatively higher selection pressure.

### Probability of V-Trajectories

This disclosure defines a v-trajectory as a frequency trajectory that decreases, then increases, as in the shape of the letter ‘v’. V-trajectories in true population frequencies may be not possible under an assumed model of asexual natural selection as disclosed herein. This disclosure will provide a mathematical proof of this statement in the later sections and use this property to evaluate how well real-world datasets adhere to the disclosed model's assumptions, which can be an important tool in evaluating the trustworthiness of inferred fitness values for decision-making.

Denote a triplet of rounds 1, 2, 3 without loss of generalization and consider variant counts c₁, C₂, c₃, total counts N₁, N₂, N₃ and true population frequencies p₁, p₂, p₃. Particular embodiments may compute the probability of a v-trajectory given count data p(p₁>p₂, p₂<p₃|c, N) under a binomial noise model cₜ~Bin(pₜ, Nₜ) as:

$${\int }_{0}^{1} BetaPDF ( {p}_{2} ; {c}_{2} , {N}_{2} - {c}_{2} ) \cdot ( 1 - BetaCDF ( {p}_{2} ; {c}_{1} , {N}_{1} - {c}_{1} ) ) \cdot ( 1 - BetaCDF ( {p}_{2} ; {c}_{3} , {N}_{3} - {c}_{3} ) ) \cdot {dp}_{2}$$

Given count data at more than three rounds, particular embodiments may take the probability a variant has a v-trajectory given all count data, as the maximum probability of a v-trajectory among any triplet of consecutive rounds. To report the fraction of a population comprised of variants violating assumed dynamics, particular embodiments may sum the read counts of all variants with probability of v-trajectory above a threshold, set at 95% in this disclosure.

### Method Comparison

This section discusses differences between ACIDES, Enrich2, and EVFI.

Enrich2 may not properly account for non-linear effects at three or more timepoints. Enrich2 is primarily designed for deep mutational scanning which compares enrichment of mutants around a wild-type sequence. Its method differs based on whether there is a designated wild-type sequence or not, and Enrich2 may assume that the wild-type sequence is observed in every timepoint. In general, directed evolution may act on a diverse input population that has not clearly designated wild-type sequence, so this disclosure studies the procedure that Enrich2 uses for datasets with three or more timepoints lacking a wild-type sequence. In the notation of this disclosure, Enrich2 performs regression for a variant g on a paired dataset (t, y(t))^{T}, where the regression target y(t) is:

$$y ( t ) {=}^{△} log ( \frac{\frac{1}{2} + {C}_{g , t}}{\frac{1}{2} + {\sum }_{{g}^{′}} {C}_{{g}^{′} t}} ) ≃ log ( {P}_{g , t} )$$

where ½ acts as a pseudocount, and in the second line removing the pseudo count Enrich2 may effectively regress on log variant frequency, taking the regression slope as the variant's score.

Enrich2 may assume that variant score does not vary by time, which means it assumes the same dynamics in equation (4) which used in EVFI. However, Enrich2's method for score regression may contradict this premise: in the noiseless setting, its regression score is not consistent with ω in equation (4).

Taking the log of equation (4), we get:

$$log {p}_{g , t + 1} - log {p}_{g , t} = log {w}_{g} - log {\sum }_{{g}^{′}} {w}_{{g}^{′}} {p}_{{g}^{′} , t} .$$

Enrich2's regression slope may effectively capture the right-hand side, which includes the term log ΣZ_{g′}w_{g′}p_{g′,t} which is a function of t. For the regression slope to correspond to log fitness, it would have to be equivalent to log w_{g}, but it is not.

This shows that despite Enrich2 assuming the dynamics in equation (4), and despite aiming to infer a single enrichment score corresponding to fitness, its method may be not correct in inferring a score that corresponds to fitness.

ACIDES has poor behavior on variants with many zero counts. Consider a variant which has zero read count in every timepoint except the last time-point, where it has a read count of one. ACIDES may assume all variants are present at all timepoints, and infer an initial abundance ρ_{g,t=1} for each variant g at the first timepoint, and a fitness score a_{g} representing the rate of growth under selection pressure.

ACIDES may model the expected frequency of variant g at time t as:

$${g}^{′} {ρ}_{g , t} = \frac{{ρ}_{g , t = 1} exp ( {a}_{g} t )}{{\sum }_{g}^{′} {ρ}_{{g}^{′} , t = 1} exp ( {a}_{g} , t )}$$

which may be recognized as the continuous-time version of equation (4) when a_{g} corresponds to log fitness. ACIDES may learn a_{g},ρ_{g,t=1} from count data by maximum likelihood.

For a variant which has zero read count in all timepoints except the last timepoint, the maximum likelihood solution may push initial frequency as close to zero as possible and push the growth rate a_{g} to be as high as possible. This may push the expected frequencies as close to zero as possible for all timepoints other than the last timepoint. This can cause ACIDES to infer excessively high scores for these variants which have little observed data.

Variants like these were observed in multiple datasets in this disclosure. Such variants are therefore not uncommon.

ACIDES' rank robustness metric can assist in decision-making for variant prioritization and serves to add large error bars and uncertainty around the score estimates for such variants. However, error estimates and confidence intervals may be constructed using a Gaussian approximation to the likelihood function centered at the maximum likelihood estimate. This means that even though score estimates can have high uncertainty, it may be symmetric around artificially high score estimates.

For evaluation, ACIDES was run with default settings. Enrich2 was run with the ‘full’ argument and WLS scoring for datasets with three or more timepoints, and ratios scoring for datasets with two timepoints. Two-timepoint enrichment was computed for two timepoints t, t′ for a variant g with a pseudo count of 0.5 as:

$${( \frac{{C}_{g , {t}^{′}} + 0.5}{{C}_{g , t} + 0.5} )}^{1 / ( {t}^{′} - t )} \qquad ( 12 )$$

where the power adjustment scales the enrichment between two timepoints which can be separated by several rounds, to the effective enrichment per round.

Each method's inferred growth rate was used to simulate forward in time all variants present in the second-to-last timepoint population, to the final timepoint, to obtain predicted final timepoint frequencies. This evaluation strategy was chosen as it focuses evaluation on the quality of inferred fitness scores.

Noise-weighted Pearson correlation of predicted enrichment to observed enrichment from time t to t+1 is computed with weights for each variant as:

$$\frac{1}{\frac{1}{{C}_{g , t} + 0.5} + \frac{1}{{C}_{g , t + 1} + 0.5}} \qquad ( 13 )$$

which is motivated as the inverse of the variance of the observed enrichment under Poisson assumptions.
Adjusting Inferred Fitness with Off-Target Data

A common setting may include availability of directed evolution data for a single population evolved in (a) a campaign against the target of interest and the instrument (target+instrument), and also (b) a campaign only against the instrument (instrument only). For example, a target protein may be immobilized on beads (representing the instrument), and the evolved drugs are washed over the bead-bound immobilized target protein, during the (target+instrument) campaign. Separately, the population can be split and washed only over the beads, without the target protein present, to attempt to adjust for general stickiness. With such data, two enrichment scores or fitness can be estimated: a (target+instrument) fitness, and an (instrument-only) fitness.

These values may be commonly combined by dividing the (target+instrument) fitness by the (instrument-only) fitness: genotype variants with a high ratio score thus have relatively higher (target+instrument) fitness, and relatively lower (instrument-only) fitness, making them good candidates for variant nomination and further testing.

However, this approach may fail to consider the identifiability up to proportionality property of fitness. As two fitness inference procedures were performed, the relative scale between the two inferred fitness may be unrelated and unknown. For example, it may be that no genotype variant has any meaningful off-target activity, or that all genotype variants do. These two scenarios would lead to dramatically different decisions in practice: perhaps the entire population is not good, or perhaps one can mine deeper into the population than otherwise expected. These scenarios can be distinguished by measuring the total absolute abundance of the population: if it decreases dramatically when washing only over beads, then the population may have little off-target activity. However, DNA sequencing data of population frequencies may not provide such information. Conventional work and common practice may have failed to appreciate this rescaling issue, which may lead to suboptimal decision-making.

This disclosure proves an upper-bound as a partial solution to this rescaling problem under the assumption that (target+instrument) fitness is a sum of (target-only) fitness and (instrument-only) fitness. This upper bound on the rescaling factor may provide a simple, principled method to correct fitness for off-target activity and distinguish between populations with high or low aggregate off-target activity, enabling scientists to mine deeper or choose to discard populations.

Theorem 1 (Upper bound for adjusting off-target fitness). Suppose, from inferred relative fitness c₁wₜₒₜₐₗ and c₂w_{off} which are proportional by unknown constants c₁, c₂ to absolute fitness ω as c₁ωₜₒₜₐₗ=ωₜₒₜₐₗ and c₂w_{off}=ω_{off}. Further, suppose that

$${ω}_{t o t a l} = {ω}_{o n} + {ω}_{o f f} . \qquad ( 14 )$$

Rewritten, we have:

$${\underbrace{{c}_{1} {w}_{total}}}_{available} = {\underbrace{{c}_{1} {w}_{on}}}_{goal} + {\underbrace{\frac{{c}_{1}}{{c}_{2}}}}_{unknown} {\underbrace{{c}_{2} {w}_{off}}}_{available} \qquad ( 15 )$$

### Then,

$${arg min}_{g} \frac{{c}_{1} {w}_{total , g}}{{c}_{2} {w}_{off , g}} \geq \frac{{c}_{1}}{{c}_{2}} . \qquad ( 16 )$$

Proof Using the property that absolute fitness must be non-negative,

$${c}_{1} {w}_{t otal} = {c}_{1} {w}_{o n} + \frac{{c}_{1}}{{c}_{2}} {c}_{2} {w}_{o f f} \qquad ( 17 )$$

$${c}_{1} {w}_{t otal} \geq 0 + \frac{{c}_{1}}{{c}_{2}} {c}_{2} {w}_{o f f} \qquad ( 18 )$$

Dividing through, for each element g we have:

$$\frac{{c}_{1} {w}_{total , i}}{{c}_{2} {w}_{off , i}} \geq \frac{{c}_{1}}{{c}_{2}} . \qquad ( 19 )$$

### Probability of Improved Fitness

Particular embodiments may estimate the probability that variant g has higher inferred fitness than variant r given count data:

$$p ( {w}_{g} > {w}_{r} \vert C ) = \frac{p ( C \vert {w}_{g} , {w}_{r} ) p ( {w}_{g} , {w}_{r} ) 1 ( {w}_{g} > {w}_{r} )}{p ( C )} \qquad ( 20 )$$

where 1(⋅) is the indicator function.

Particular embodiments may take p(C|w_{g}, wᵣ) to follow a Dirichlet-Multinomial distribution, so that:

$${mask}_{t} ( {C}_{t + 1} ) ∼ Dirichlet - Multinomial ( α = \hat{α} {mask}_{t} ( \frac{w}{{w}^{T} {C}_{t}} ⊙ {C}_{t} ) ) \qquad ( 21 )$$

$$where$$

$${\hat{p}}_{t + 1} {=}^{△} \frac{w}{{w}^{T} {C}_{t}} ⊙ {C}_{t} \qquad ( 22 )$$

Particular embodiments may use a uniform prior for p(w_{g}, wᵣ) and learn â from data by maximum likelihood jointly with fitness inference. To efficiently estimate p(w_{g}>wᵣ|C), particular embodiments may use the grouping property of the Dirichlet-Multinomial distribution to group counts into three bins: counts for variant g, for variant r, and summed counts for all variants other than g, r which this disclosure denotes o, whose likelihood are governed by three fitness variables: w_{g}, wᵣ, wₒ. Particular embodiments may estimate p(w_{g}>wᵣ|cᵣ, c_{g}, cₒ) using Monte Carlo estimation. To address the non-identifiability property, particular embodiments may compute likelihoods using uniformly distributed random samples for w_{g}, wᵣ while keeping wₒ fixed. The details are described as follows.

Computing p(C|w_{g}, wᵣ) which marginalizes over the fitness values of all variants besides g, r can be a challenging problem involving very high-dimensional integration in the typical case where the number of unique genotype variants is 30,000. However, the aggregation property of the Dirichlet-Multinomial distribution may hold that if (x₁, . . . , xₖ)~DM(α₁, . . . , αₖ), then for any indices i,j, (x₁, . . . , xᵢ+xⱼ, . . . , xₖ)~DM(α₁, . . . , αᵢ+αⱼ, . . . , αₖ). This disclosure uses this property to construct a likelihood for w_{g}, wᵣ that is easier to compute by aggregating variants into three bins: g, r, and all other variants denoted as o for “other”. Aggregation may be performed by summation, so that

$${c}_{o , t} {=}^{△} {\sum }_{i ∉ \{ g , r \}} {c}_{i , t} \qquad ( 23 )$$

Dropping the mask notation for ease of exposition, and applying aggregation, yields:

$$p ( {c}_{g , t} , {c}_{r , t} , {c}_{o , t} \vert {w}_{g} , {w}_{r} , {w}_{o} ) = Dirichlet - Multinomial ( α = \hat{α} ( {\hat{p}}_{g , t} , {\hat{p}}_{r , t} , {\hat{p}}_{o , t} ) ) \qquad ( 24 )$$

As predicted frequencies \hat{p} are a function of count data in the previous timepoint, the aggregated likelihood is a product over all timepoints:

$$p ( {c}_{g} , {c}_{r} , {c}_{o} \vert {w}_{g} , {w}_{r} , {w}_{o} ) = {\prod }_{t = 2}^{T} p ( {c}_{g , t} , {c}_{r , t} , {c}_{o , t} \vert {w}_{g} , {w}_{r} , {w}_{o} ) \qquad ( 25 )$$

As this likelihood can be invariant to scaling w_{g}, wᵣ, wₒ, together by any multiplicative constant (See, Property 2), this disclosure holds wₒ fixed. This disclosure uses N uniformly distributed random samples of w_{g}, wᵣ to estimate:

$$p ( {w}_{g} > {w}_{r} \vert C ) \approx \frac{{\sum }_{i = 1}^{N} p ( {c}_{g} , {c}_{r} , {c}_{o} \vert {w}_{g , i} , {w}_{r , i} , {w}_{o} ) 1 ( {w}_{g , i} > {w}_{r , i} )}{{\sum }_{i = 1}^{N} p ( {c}_{g} , {c}_{r} , {c}_{o} \vert {w}_{g , i} , {w}_{r , i} , {w}_{o} )} \qquad ( 26 )$$

This disclosure handles masking in the computational implementation of this approach.

### Mathematical Properties of Evolutionary Fitness Inference

This section presents mathematical properties of the data generative process and evolutionary fitness inference. We begin by restating the disclosed simple model of growth under asexual natural selection from equation (1):

$${n}_{g , t + 1} = {ω}_{g} , {n}_{g , t} \qquad ( 1 )$$

Property 1 (Non-linear population frequency dynamics). Suppose a population follows the dynamics of equation (1). Then, population frequency

$${p}_{g , t} {=}^{△} {n}_{g , t} / {\sum }_{g}^{′} {n}_{{g}^{′} t}$$

obeys:

$${p}_{g , t + 1} = \frac{{w}_{g}}{{\sum }_{j} {w}_{j} {p}_{j , t}} {p}_{g , t} \qquad ( 4 )$$

or equivalently, written in bold vector notation,

$${p}_{t + 1} = \frac{w}{{w}^{T} {p}_{t}} ⊙ {p}_{t} \qquad ( 27 )$$

where ⊙ means element-wise multiplication.

Proof Using Nₜ₊₁=Σ_{g} n_{g,t+1}=Σ_{g} w_{g}n_{g,t}, we have

$${n}_{g , t + 1} = {w}_{g} {n}_{g , t}$$

$$\frac{{n}_{g , t + 1}}{{N}_{t + 1}} = \frac{{w}_{g}}{{N}_{t + 1}} {n}_{g , t} ( divide by {N}_{t + 1} )$$

$${p}_{g , t + 1} = \frac{{w}_{g}}{{\sum }_{j} {w}_{j} {n}_{j , t}} {n}_{g , t} ( replace with definitions )$$

$${p}_{g , t + 1} = \frac{{N}_{t} {w}_{g}}{{\sum }_{j} {w}_{j} {n}_{j , t}} \frac{{n}_{g , t}}{{N}_{t}} ( multiply by {N}_{t} / {N}_{t} )$$

$${p}_{g , t + 1} = \frac{{w}_{g}}{{\sum }_{j} {w}_{j} \frac{{n}_{t , j}}{{N}_{t}}} {p}_{g , t} ( move {N}_{t} through fraction )$$

$${p}_{g , t + 1} = \frac{{w}_{g}}{{\sum }_{j} {w}_{j} {p}_{j , t}} {p}_{g , t} ( replace with definitions )$$

Remark. In vector notation, w, pₜ, pₜ₊₁ are all G-dimensional vectors where pₜ, pₜ₊₁ have positive entries summing to 1, and w has positive entries.

Thus far this disclosure has considered discrete time dynamics. The continuous time analogue, for time update Δt, may be:

$${p}_{t + △ t} = \frac{exp ( △ t log ( w ) )}{{exp ( △ t log ( w ) )}^{T} {p}_{t}} ⊙ {p}_{t} \qquad ( 28 )$$

Property 2 (Fitness scale invariance, or identifiability up to proportionality). In equation (27), multiplying w by a positive constant factor c for a given pₜ does not change pₜ₊₁.

Proof

$$\begin{matrix}{p}_{t + 1} = \frac{c w}{{( c w )}^{T} {p}_{t}} ⊙ {p}_{t} \\ = \frac{c w}{c {w}^{T} {p}_{t}} ⊙ {p}_{t} \\ = \frac{w}{{w}^{T} {p}_{t}} ⊙ {p}_{t}\end{matrix}$$

Remark. Property 2 means that given a count table, the scale of fitness values cannot be determined, so only the relative ratio between any two fitness values is meaningful. If fitness values are not explicitly scaled during training, then it should be expected that running fitness inference multiple times on the same dataset will give differently scaled fitness values. This may be an essential property of fitness inference with significant impact on interpreting and using fitness values for downstream applications.

Property 3 (Population dynamics also hold for population subsets). Suppose w, pₜ, pₜ₊₁ are G-dimensional vectors for G genotype variants that obey equation (27). For any m-dimensional subset of these genotype variants with associated subset vectors wₛ, p_{s,t}, p_{s,t+i} (where p_{s,t}, p_{s,t+1} do not sum to one, as they are subsets of a frequency vector), denote

$${\tilde{p}}_{s , t} {=}^{△} {p}_{s , t} / ( {\sum }_{j = 1}^{m} {p}_{s , t , j} )$$

as the re-normalized m-dimensional vector of frequencies of the subset. Then, one may have:

$${\tilde{p}}_{s , t + 1} = \frac{{w}_{s}}{{w}_{s}^{T} {\tilde{p}}_{s , t}} ⊙ {\tilde{p}}_{s , t} \qquad ( 28 )$$

Proof Without loss of generality, suppose w, pₜ, pₜ₊₁ are ordered such that the subset s corresponds to the first m elements of each. One may have for each i where 1≤i≤m:

$${p}_{t + 1 , i} = \frac{{w}_{i}}{{\sum }_{j = 1}^{G} {w}_{j} {p}_{t , j}} {p}_{t , i}$$

$$\frac{{p}_{t + 1 , i}}{{\sum }_{k = 1}^{m} {p}_{t + 1 , k}} = \frac{{w}_{i}}{{\sum }_{j = 1}^{G} {w}_{j} {p}_{t , j}} {p}_{t , i} ( \frac{1}{{\sum }_{k = 1}^{m} {p}_{t + 1 , k}} )$$

$$\frac{{p}_{t + 1 , i}}{{\sum }_{k = 1}^{m} {p}_{t + 1 , k}} = \frac{{w}_{i}}{{\sum }_{j = 1}^{G} {w}_{j} {p}_{t , j}} {p}_{t , i} ( \frac{{\sum }_{j = 1}^{G} {w}_{j} {p}_{t , j}}{{\sum }_{k = 1}^{m} {w}_{k} {p}_{t , k}} )$$

where this disclosure used

$${\sum }_{k = 1}^{m} {p}_{t + 1 , k} = {\sum }_{k = 1}^{m} \frac{{w}_{k}}{{\sum }_{j = 1}^{G} {w}_{j} {p}_{t , j}} {p}_{t , k} .$$

Cancelling the term

$${\sum }_{j = 1}^{G} {w}_{j} {p}_{t , j} ,$$

one may obtain:

$$\begin{matrix}\frac{{p}_{t + 1 , i}}{{\sum }_{k = 1}^{m} {p}_{t + 1 , k}} = \frac{{w}_{i}}{{\sum }_{k = 1}^{m} {w}_{k} {p}_{t , k}} {p}_{t , i} \\ = \frac{{w}_{i}}{{\sum }_{k = 1}^{m} {w}_{k} \frac{{p}_{t , k}}{{\sum }_{z = 1}^{m} {p}_{t , z}}} \frac{{p}_{t , i}}{{\sum }_{k = 1}^{m} {p}_{t , k}} \\ {\tilde{p}}_{t + 1 , i} = \frac{{w}_{i}}{{\sum }_{k = 1}^{m} {w}_{k} {\tilde{p}}_{t , k}} {\tilde{p}}_{t , k}\end{matrix}$$

Property 4 (Frequency-weighted fitness increases monotonically over time). Suppose w and pₜ for rounds t are C-dimensional vectors for G genotype variants that obey equation (27). For any t, one may have:

$${w}^{T} {p}_{t + 1} \geq {w}^{T} {p}_{t} \qquad ( 29 )$$

Proof By equation (27), one may have:

$${P}_{g , t + 1} = \frac{{w}_{g}}{{\sum }_{j}^{} {w}_{j} {p}_{j , t}} {p}_{g , t}$$

Intuitively, consider three cases: for any variant indexed g:

$${w}_{g} > {w}^{T} {p}_{t} : Then \frac{{w}_{g}}{{\sum }_{j}^{} {w}_{j} {p}_{j , t}} > 1 , and {p}_{g , t + 1} > {p}_{g , t}$$

$${w}_{g} > {w}^{T} {p}_{t} : Then \frac{{w}_{g}}{{\sum }_{j}^{} {w}_{j} {p}_{j , t}} = 1 , and {p}_{g , t + 1} = {p}_{g , t}$$

$${w}_{g} > {w}^{T} {p}_{t} : Then \frac{{w}_{g}}{{\sum }_{j}^{} {w}_{j} {p}_{j , t}} < 1 , and {p}_{g , t + 1} < {p}_{g , t}$$

Because pₜ₊₁ and pₜ both sum to 1, the net effect is pₜ₊₁ places higher weight on all w_{g} greater than w^{T}pₜ, relative to pₜ. It places lower weight on all w_{g} less than w^{T}pₜ. And it places the same weight (p_{g,t+1}=p_{g,t}) on all w_{g}=w^{T}pₜ. Thus, one can intuitively see that w^{T}pₜ₊₁≥w^{T}pₜ.
More formally, this disclosure rewrites the lemma, expressing pₜ₌₁ in terms of pₜ:

$${\sum }_{g} {w}_{g} \frac{{w}_{9} {p}_{g , t}}{{\sum }_{j}^{} {w}_{j} {p}_{j , t}} \geq {\sum }_{g} {w}_{g} {p}_{g , t}$$

For clarity of notation, one can drop t moving forward:

$${\sum }_{i} {w}_{i}^{2} {p}_{i} \geq {( {\sum }_{i} {w}_{i} {p}_{i} )}^{2}$$

$${\sum }_{i} {w}_{i}^{2} {p}_{i} \geq {\sum }_{i} {w}_{i}^{2} {p}_{i}^{2} + {\sum }_{j}^{G} {\sum }_{k > j}^{G} 2 {w}_{j} {w}_{k} {p}_{j} {p}_{k}$$

$${\sum }_{i} {w}_{i}^{2} {p}_{i} ( 1 - {p}_{i} ) \geq {\sum }_{j}^{G} {\sum }_{k > j}^{G} 2 {w}_{j} {w}_{k} {p}_{j} {p}_{k}$$

$${\sum }_{i} ( {w}_{i}^{2} {p}_{i} ( {\sum }_{j \neq i} {p}_{j} ) ) \geq {\sum }_{j}^{G} {\sum }_{k > j}^{G} 2 {w}_{j} {w}_{k} {p}_{j} {p}_{k}$$

$${{\sum }^{}}_{j} {{\sum }^{}}_{k > j} {w}_{j}^{2} {p}_{j} {p}_{k} + {w}_{k}^{2} {p}_{j} {p}_{k} \geq {\sum }_{j}^{G} {\sum }_{k > j}^{G} 2 {w}_{j} {w}_{k} {p}_{j} {p}_{k}$$

$${{\sum }^{}}_{j} {\sum }_{k > j} {w}_{j}^{2} {p}_{j} {p}_{k} - 2 {w}_{j} {w}_{k} {p}_{j} {p}_{k} + {w}_{k}^{2} {p}_{j} {p}_{k} \geq 0$$

$${\sum }_{j} {\sum }_{k > j} {p}_{j} {{p}_{k} ( {w}_{j} - {w}_{k} )}^{2} \geq 0$$

which is true, as pᵢ>0 for all i by definition.

Property 5 (V-trajectories are not possible: variant frequencies cannot decrease then increase). Suppose w and pₜ for rounds t are G-dimensional vectors for G genotype variants that obey equation (27). Then, for any variant g and consecutive time rounds t<t′<t″, it cannot be true that p_{g,t}>p_{g,t′} and p_{g,t′}<p_{g,t″}.

Proof. This disclosure uses the property that frequency-weighted fitness w^{T}pₜ increases monotonically over time (equation (29)). As

$${p}_{g , t + 1} = \frac{{w}_{g}}{{\sum }_{j} {w}_{j} {p}_{j , t}} {p}_{g , t}$$

the rate of change in frequency

$$\frac{{w}_{g}}{\sum {jw}_{j} {p}_{j , t}^{-}}$$

decreases monotonically over time, making the frequency trajectory a concave function over time.

Remark. This theorem states that under a simple model of exponential growth, variant frequencies cannot decrease then increase (a “v-trajectory”). However, real-world datasets can have v-trajectories for a variety of reasons, such as changes in selection pressure (i.e., changing targets). If a population undergoes extreme bottlenecking between some rounds, then v-trajectories can occur due to high-variance genetic drift. It is possible for the frequency-weighted fitness of a population to decrease if many low-fitness variants are introduced into a population, which can induce v-trajectories when analyzing the population as whole. This particular issue can be avoided during data analysis by instead searching for v-trajectories for a query variant within a restricted subset of variants that all first appear in the population at the same time or earlier than the first appearance of the query variant.

### Mutation Rates in In Vitro Display Assays

This section discusses the possible rates of mutation that may occur in three common in vitro display protocols: yeast surface display, phage display, and mRNA display. This disclosure expresses mutation rates in units of mutations per nucleotide per replication, which this disclosure abbreviates as mut/nt/rep.

A common protocol for phage display may use M13 filamentous phage replicating inside E. coli. M13 uses the host E. coli DNA polymerase to replicate its phage genome. E. coli DNA polymerase replicating the E. coli genome may have an overall mutation rate of 10⁻¹⁰ mut/nt/rep. However, M13 replicating using E. coli DNA polymerase has been observed to have 1000× higher mutation rate, at 10-⁷ mut/nt/rep. This fits Drake's rule, at 0.003 muts/genome/rep.

In general, DNA replication can be a complex process involving DNA polymerase but also multiple DNA repair pathways involving a variety of proteins. E. coli's replication mutation rate of 10-¹⁰ mut/nt/rep can be attributed to three mechanisms: base selection with 10⁵-fold reduction in mutation rate, mismatch repair with 10³-fold reduction, and proofreading with 10²-fold reduction. This E. coli's high-fidelity replication may be attributable not just to the use of E. coli DNA polymerase, but many other DNA repair proteins.

In principle, it is not clear that M13 phage replicating using E. coli DNA polymerase would also benefit from the same DNA repair machinery. Conventional work has observed that the phage genome is depleted for GATC motifs which contribute to methylation-induced mismatch repair in E. coli, which may explain increased observed mutation rates in phage.

Furthermore, phage generation time can be relatively fast, with M13 modeled at 30 minutes per generation. In practice, many generations of phage replication might occur between each selection round in phage display, which increases the effective mutation rate. For instance, if phage is left to replicate in E. coli overnight (12 hours) such that M13 phage replicate 24 times, the rate of 10-⁷ mut/nt/rep may become 2.4×10-⁶ mut/nt/24-reps.

Extending this to an evolving gene variant with 100 nucleotides and a library size of 10⁹ molecules, about 10⁵ mutations may be introduced across the population every 12 hours of phage replication on average.

In mRNA display, polymerase chain reaction (PCR) can be used between selection rounds to amplify the population. An example High-Fidelity DNA polymerase has a mutation rate of 4.4×10-⁷ mut/nt/rep. While the number of PCR cycles used in practice can vary, over 30 replication cycles, this may become 1.3×10-⁵ muts/nt. If the evolving peptide or gene has a length of 100 nucleotides, and the population has 10¹³ molecules in it, a mutation rate of 1.3×10-⁵ muts/nt means 10¹⁰ (ten billion) mutations are introduced across the population each round, on average.

Not using a high-fidelity DNA polymerase can raise mutation rates significantly. Taq DNA polymerase has a mutation rate of 2.28×10-⁵ mut/nt/rep, which is nearly 100 times higher than the aforementioned example High-Fidelity DNA Polymerase.

A common protocol for yeast surface display may use pCTcon2 plasmid in Saccharomyces cerevisiae. The pCTcon2 plasmid is a low copy number plasmid typically with one copy per yeast cell, which helps to ensure an accurate connection between the phenotype that selection acts upon, and the genotype that induces the phenotype. pCTcon2 is a centromeric plasmid which replicates like an additional yeast chromosome. Diploid S. cerevisiae genome replication occurs at 10-¹⁰ mut/nt/rep, while haploid genome replication has been reported at 10-⁹ mut/nt/rep. pCTcon2 functioning as a single-copy chromosome may be expected to inherit the haploid mutation rate.

Yeast doubling time is about 90 minutes. If yeast are grown for 24 hours which is 16 generations, the 10-⁹ mut/nt/rep may become 1.6×10-⁸ mut/nt/24 hr.

With an evolving gene length of 100 nucleotides and a library size of 10⁸, a mutation rate of 1.6×10-⁸ mut/nt/24 hr means that 100 mutations are introduced across the population each round, on average.

Physical stress can cause mutations. Furthermore, there can be significant variation in mutation rates: hot spots and cold spots in the genome can vary by up to 10×. Environment and growth conditions can also impact mutation rates, by up to 3-fold.

### Fitness Inference Versus Computing Round-Over-Round Enrichment

In the disclosed framework, if the data comprises two timepoints, fitness inference may be conceptually identical to computing round-over-round enrichment.

Round-over-round enrichment for a variant g is computed as:

$$enrichment ( g ) = \frac{{p}_{g , t = 1}}{{p}_{g , t = 0}} ,$$

so the enrichment ratio comparing two variants g, r is:

$$\frac{enrichment ( g )}{enrichment ( g )} = \frac{{p}_{g , t = 1}}{{p}_{g , t = 0}} \frac{{p}_{r , t = 0}}{{p}_{r , t = 1}} .$$

In the disclosed framework, fitness dynamics on variant frequencies may follow

$${p}_{g , t +} = \frac{{ω}_{g}}{{\sum }_{j} {ω}_{j} {p}_{j , t}} {p}_{g , t} ,$$

so the fitness ratio comparing two variants g, r is:

$$\frac{{ω}_{g}}{{ω}_{r}} = \frac{{p}_{g , t = 1}}{{p}_{g , t = 0}} \frac{{p}_{r , t = 0}}{{p}_{r , t = 1}} .$$

This relationship can be true for any choice of two variants, so it holds for all variants.

Here, this section discusses the differences between fitness inference and round-over-round enrichment.

The primary difference may be that fitness inference provides a framework and method that supports three or more timepoints, while round-over-round enrichment requires pre-specifying two timepoints. When T timepoints are available, there are

$$( \begin{matrix}n \\ k\end{matrix} )$$

ordered timepoint pairs that can be used to compute different round-over-round enrichments. For a typical T=5, there are 10 timepoint pairs: 1→5, 2→5, . . . , 4→5. In general, enrichment computed using different timepoint pairs may disagree with each other—some variants may score highly using certain timepoints pairs, but not others—making it unclear how to make decisions to nominate variants.

The final timepoint may represent the most competitive selection environment, so in the above example, 4→5 would be the best timepoint pair to compute enrichment to identify variants with highest fitness. However, by using all available timepoints, fitness inference can denoise enrichment scores. For example, two variants might have an enrichment ratio of 2 in the final timepoint pair, but enrichment ratios of 1.5 and 1.7 in earlier rounds. Only using round-over-round enrichment would only use one of these values, while fitness inference would effectively average over these observed enrichment ratios to estimate a denoised version of enrichment.

With round-over-round enrichment, it may be unclear what to do when the input read count for a variant is zero, as it is not possible to divide by zero. Some conventional methods add pseudo counts to treat zeros as very small numbers, but this can introduce a significant source of bias in follow-up decision-making with little supporting evidence.

EVFI may be capable of inferring fitness for any variant as long as it has non-zero counts in at least one pair of consecutive timepoints. Furthermore, EVFI may infer fitness for all such variants on the same scale.

Suppose a variant has read count data over 5 timepoints: [0, 0, 0, 3, 10], while another variant has [5, 3, 0, 0, 0]. Under very mild conditions—there may be a “bridge” of variants that compete with each other, that connects the first two timepoints to the last timepoints—fitness inference may infer fitness of both variants and enable comparing these two variants on the same scale.

In contrast, round-over-round enrichment may only be able to compute a meaningful, data-driven enrichment score for each variant in one out of ten possible timepoint pairs and cannot compute enrichment scores to compare the two variants.

Because of EVFI's ability to handle zeros in this manner, the size of the variant set with inferred fitness may always be equal to or greater than the size of the variant set with data-driven enrichment scores for any timepoint pair.

Constructing Diverse Variant Sets with High Fitness

In particular embodiments, the molecule nomination system 190 may process a plurality of biomolecule representations associated with a plurality of respective second biomolecules by the machine-learning model (e.g., DeepEVFI) to determine a plurality of inferred fitness scores for the plurality of second biomolecules, respectively. The molecule nomination system may further select, based on the inferred fitness scores for the plurality of second biomolecules and pairwise distances between the plurality of second biomolecules, one or more diverse biomolecules from the plurality of second biomolecules. The one or more diverse biomolecules may meet a predetermined criteria for selection.

In particular embodiments, the molecule nomination system 190 may process a plurality of biomolecule representations associated with a plurality of respective second biomolecules by the machine-learning model to determine a plurality of inferred fitness scores for the plurality of second biomolecules, respectively. The molecule nominations system may then generate one or more clusters for the plurality of biomolecule representations based on a clustering algorithm. The molecule nomination system 190 may further select one or more diverse biomolecules from the plurality of second biomolecules by identifying the one or more diverse biomolecules from the one or more clusters, respectively, wherein each of the one or more diverse biomolecules is associated with a top inferred fitness score in the respective cluster.

Particular embodiments may use a greedy algorithm that iteratively adds elements to a nomination set. Consider a set of elements X, a score function such as log fitness f(x), and a distance function d(x, x′). The nomination set S can be initialized with the variant with the highest log fitness, and a weight α is chosen. At each iteration, all variants not in the set may be scored using:

$$a f ( x ) + ( 1 - a ) \frac{1}{\vert S \vert} {\sum }_{{x}^{′} ϵ S} d ( x , {x}^{′} ) \qquad ( 30 )$$

and the highest-scoring variant may be added to the set. The iteration may repeat until the set size reaches the desired number. To plot the Pareto frontier of nomination sets trading off mean log fitness and mean pairwise distance, particular embodiments vary α from 0 to 1. For all datasets other than M-MP, particular embodiments use one-hot encoded vectors and L2 distance. For M-MP, particular embodiments use chirality-aware Morgan fingerprint representations with radius 3 and nBits 2048, and compute distance as 1-Tanimoto similarity. To make UMAP plots, one-hot encoded vectors were transformed with principal component analysis into up to 50 dimensions, then UMAP was applied.

### FGFR1 Variant Nomination

The embodiments disclosed herein used EVFI on three tracks: ABCDE, ABCDF, and ABCDH, using the population names described in the methods section on yeast display on FGFR1. These tracks were chosen as populations E, F, and H underwent selection against both D3b and D3c for the entirety of their selection history. EVFI was used to infer fitness and nominate variants separately for each track and pooled together. In the disclosed study of the correlation between fitness and affinity, the embodiments use the fitness scores from the ABCDH track which had the strongest selection pressure.

### TEAD Variant Nomination

To nominate variants, particular embodiments trained DeepEVFI on the TEAD data, and used to score the peptides. Then, a variational autoencoder was trained on macrocyclic peptides represented with one-hot encoding and Morgan fingerprint using reconstruction loss. The encoder was used to encode the 5000 peptides with highest DeepEVFI score, and uniform manifold approximation and projection (UMAP) was used to cluster the latent embeddings into 150 clusters. The top-scoring peptide was chosen from each cluster to yield 150 variants, then the top 20 scoring peptides among the 150 were nominated for testing.

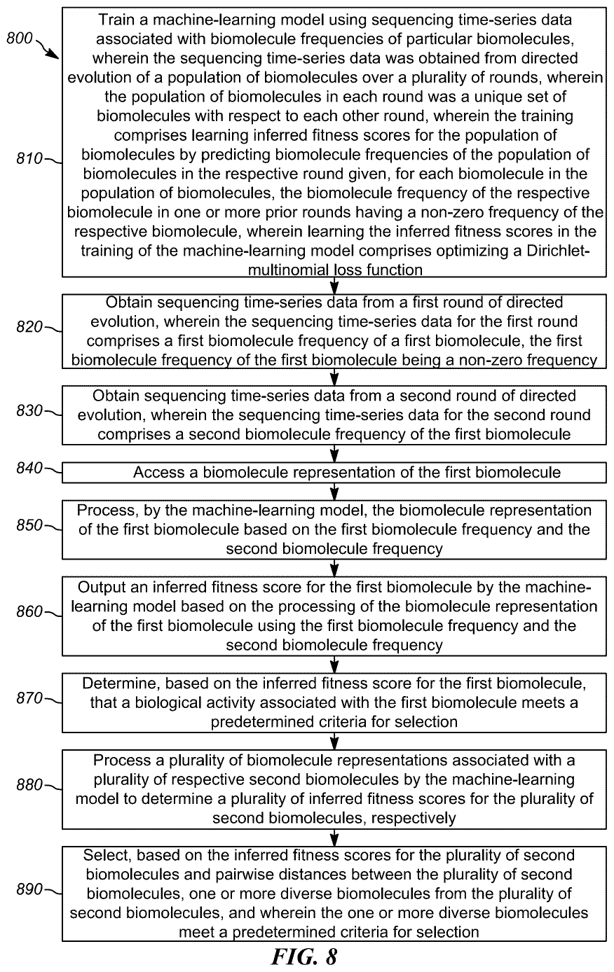

FIG. 8 illustrates an example method 800 for biomolecule fitness inference. The method may begin at step 810, where the molecule nomination system 190 may train a machine-learning model using sequencing time-series data associated with biomolecule frequencies of particular biomolecules, wherein the sequencing time-series data was obtained from directed evolution of a population of biomolecules over a plurality of rounds, wherein the population of biomolecules in each round was a unique set of biomolecules with respect to each other round, wherein the training comprises learning inferred fitness scores for the population of biomolecules by predicting biomolecule frequencies of the population of biomolecules in the respective round given, for each biomolecule in the population of biomolecules, the biomolecule frequency of the respective biomolecule in one or more prior rounds having a non-zero frequency of the respective biomolecule, and wherein learning the inferred fitness scores in the training of the machine-learning model comprises optimizing a Dirichlet-multinomial loss function. At step 820, the molecule nomination system 190 may obtain sequencing time-series data from a first round of directed evolution, wherein the sequencing time-series data for the first round comprises a first biomolecule frequency of a first biomolecule, the first biomolecule frequency of the first biomolecule being a non-zero frequency. At step 830, the molecule nomination system 190 may obtain sequencing time-series data from a second round of directed evolution, wherein the sequencing time-series data for the second round comprises a second biomolecule frequency of the first biomolecule. At step 840, the molecule nomination system 190 may access a biomolecule representation of the first biomolecule. At step 850, the molecule nomination system 190 may process, by the machine-learning model, the biomolecule representation of the first biomolecule based on the first biomolecule frequency and the second biomolecule frequency. At step 860, the molecule nomination system 190 may output an inferred fitness score for the first biomolecule by the machine-learning model based on the processing of the biomolecule representation of the first biomolecule using the first biomolecule frequency and the second biomolecule frequency. At step 870, the molecule nomination system 190 may determine, based on the inferred fitness score for the first biomolecule, that a biological activity associated with the first biomolecule meets a predetermined criteria for selection. At step 880, the molecule nomination system 190 may process a plurality of biomolecule representations associated with a plurality of respective second biomolecules by the machine-learning model to determine a plurality of inferred fitness scores for the plurality of second biomolecules, respectively. At step 890, the molecule nomination system 190 may select, based on the inferred fitness scores for the plurality of second biomolecules and pairwise distances between the plurality of second biomolecules, one or more diverse biomolecules from the plurality of second biomolecules, and wherein the one or more diverse biomolecules meet a predetermined criteria for selection. Particular embodiments may repeat one or more steps of the method of FIG. 8, where appropriate. Although this disclosure describes and illustrates particular steps of the method of FIG. 8 as occurring in a particular order, this disclosure contemplates any suitable steps of the method of FIG. 8 occurring in any suitable order. Moreover, although this disclosure describes and illustrates an example method for biomolecule fitness inference including the particular steps of the method of FIG. 8, this disclosure contemplates any suitable method for biomolecule fitness inference including any suitable steps, which may include all, some, or none of the steps of the method of FIG. 8, where appropriate. Furthermore, although this disclosure describes and illustrates particular components, devices, or systems carrying out particular steps of the method of FIG. 8, this disclosure contemplates any suitable combination of any suitable components, devices, or systems carrying out any suitable steps of the method of FIG. 8.

### Systems and Methods

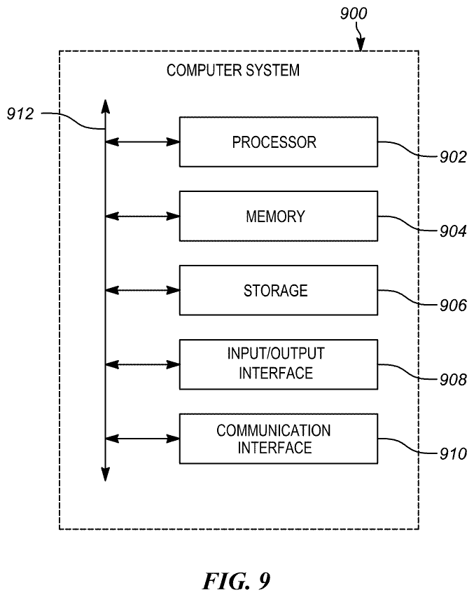

FIG. 9 illustrates an example computer system 900. In particular embodiments, one or more computer systems 900 perform one or more steps of one or more methods described or illustrated herein. In particular embodiments, one or more computer systems 900 provide functionality described or illustrated herein. In particular embodiments, software running on one or more computer systems 900 performs one or more steps of one or more methods described or illustrated herein or provides functionality described or illustrated herein. Particular embodiments include one or more portions of one or more computer systems 900. Herein, reference to a computer system may encompass a computing device, and vice versa, where appropriate. Moreover, reference to a computer system may encompass one or more computer systems, where appropriate.

This disclosure contemplates any suitable number of computer systems 900. This disclosure contemplates computer system 900 taking any suitable physical form. As example and not by way of limitation, computer system 900 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 900 may include one or more computer systems 900; be unitary or distributed; span multiple locations; span multiple machines; span multiple data centers; or reside in a cloud, which may include one or more cloud components in one or more networks. Where appropriate, one or more computer systems 900 may perform without substantial spatial or temporal limitation one or more steps of one or more methods described or illustrated herein. As an example and not by way of limitation, one or more computer systems 900 may perform in real time or in batch mode one or more steps of one or more methods described or illustrated herein. One or more computer systems 900 may perform at different times or at different locations one or more steps of one or more methods described or illustrated herein, where appropriate.

In particular embodiments, computer system 900 includes a processor 902, memory 904, storage 906, an input/output (I/O) interface 908, a communication interface 910, and a bus 912. Although this disclosure describes and illustrates a particular computer system having a particular number of particular components in a particular arrangement, this disclosure contemplates any suitable computer system having any suitable number of any suitable components in any suitable arrangement.

In particular embodiments, processor 902 includes hardware for executing instructions, such as those making up a computer program. As an example and not by way of limitation, to execute instructions, processor 902 may retrieve (or fetch) the instructions from an internal register, an internal cache, memory 904, or storage 906; decode and execute them; and then write one or more results to an internal register, an internal cache, memory 904, or storage 906. In particular embodiments, processor 902 may include one or more internal caches for data, instructions, or addresses. This disclosure contemplates processor 902 including any suitable number of any suitable internal caches, where appropriate. As an example and not by way of limitation, processor 902 may include one or more instruction caches, one or more data caches, and one or more translation lookaside buffers (TLBs). Instructions in the instruction caches may be copies of instructions in memory 904 or storage 906, and the instruction caches may speed up retrieval of those instructions by processor 902. Data in the data caches may be copies of data in memory 904 or storage 906 for instructions executing at processor 902 to operate on; the results of previous instructions executed at processor 902 for access by subsequent instructions executing at processor 902 or for writing to memory 904 or storage 906; or other suitable data. The data caches may speed up read or write operations by processor 902. The TLBs may speed up virtual-address translation for processor 902. In particular embodiments, processor 902 may include one or more internal registers for data, instructions, or addresses. This disclosure contemplates processor 902 including any suitable number of any suitable internal registers, where appropriate. Where appropriate, processor 902 may include one or more arithmetic logic units (ALUs); be a multi-core processor; or include one or more processors 902. Although this disclosure describes and illustrates a particular processor, this disclosure contemplates any suitable processor.

In particular embodiments, memory 904 includes main memory for storing instructions for processor 902 to execute or data for processor 902 to operate on. As an example and not by way of limitation, computer system 900 may load instructions from storage 906 or another source (such as, for example, another computer system 900) to memory 904. Processor 902 may then load the instructions from memory 904 to an internal register or internal cache. To execute the instructions, processor 902 may retrieve the instructions from the internal register or internal cache and decode them. During or after execution of the instructions, processor 902 may write one or more results (which may be intermediate or final results) to the internal register or internal cache. Processor 902 may then write one or more of those results to memory 904. In particular embodiments, processor 902 executes only instructions in one or more internal registers or internal caches or in memory 904 (as opposed to storage 906 or elsewhere) and operates only on data in one or more internal registers or internal caches or in memory 904 (as opposed to storage 906 or elsewhere). One or more memory buses (which may each include an address bus and a data bus) may couple processor 902 to memory 904. Bus 912 may include one or more memory buses, as described below. In particular embodiments, one or more memory management units (MMUs) reside between processor 902 and memory 904 and facilitate accesses to memory 904 requested by processor 902. In particular embodiments, memory 904 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 904 may include one or more memories 904, where appropriate. Although this disclosure describes and illustrates particular memory, this disclosure contemplates any suitable memory.

In particular embodiments, storage 906 includes mass storage for data or instructions. As an example and not by way of limitation, storage 906 may include a hard disk drive (HDD), a floppy disk drive, flash memory, an optical disc, a magneto-optical disc, magnetic tape, or a Universal Serial Bus (USB) drive or a combination of two or more of these. Storage 906 may include removable or non-removable (or fixed) media, where appropriate. Storage 906 may be internal or external to computer system 900, where appropriate. In particular embodiments, storage 906 is non-volatile, solid-state memory. In particular embodiments, storage 906 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 906 taking any suitable physical form. Storage 906 may include one or more storage control units facilitating communication between processor 902 and storage 906, where appropriate. Where appropriate, storage 906 may include one or more storages 906. Although this disclosure describes and illustrates particular storage, this disclosure contemplates any suitable storage.

In particular embodiments, I/O interface 908 includes hardware, software, or both, providing one or more interfaces for communication between computer system 900 and one or more I/O devices. Computer system 900 may include one or more of these I/O devices, where appropriate. One or more of these I/O devices may enable communication between a person and computer system 900. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 908 for them. Where appropriate, I/O interface 908 may include one or more device or software drivers enabling processor 902 to drive one or more of these I/O devices. I/O interface 908 may include one or more I/O interfaces 908, where appropriate. Although this disclosure describes and illustrates a particular I/O interface, this disclosure contemplates any suitable I/O interface.

In particular embodiments, communication interface 910 includes hardware, software, or both providing one or more interfaces for communication (such as, for example, packet-based communication) between computer system 900 and one or more other computer systems 900 or one or more networks. As an example and not by way of limitation, communication interface 910 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 910 for it. As an example and not by way of limitation, computer system 900 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 900 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 900 may include any suitable communication interface 910 for any of these networks, where appropriate. Communication interface 910 may include one or more communication interfaces 910, where appropriate. Although this disclosure describes and illustrates a particular communication interface, this disclosure contemplates any suitable communication interface.

In particular embodiments, bus 912 includes hardware, software, or both coupling components of computer system 900 to each other. As an example and not by way of limitation, bus 912 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 912 may include one or more buses 912, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (Ics) (such, as for example, field-programmable gate arrays (FPGAs) or application-specific Ics (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

### Recitation of Embodiments

Embodiment 1: A method including, by one or more computing systems: obtaining sequencing time-series data from a first round of directed evolution, wherein the sequencing time-series data for the first round comprises a first biomolecule frequency of a first biomolecule, the first biomolecule frequency of the first biomolecule being a non-zero frequency; obtaining sequencing time-series data from a second round of directed evolution, wherein the sequencing time-series data for the second round comprises a second biomolecule frequency of the first biomolecule; and outputting an inferred fitness score for the first biomolecule using the first biomolecule frequency and the second biomolecule frequency.

Embodiment 2: The method of Embodiment 1, further including: accessing a biomolecule representation of the first biomolecule; processing, by a machine-learning model, the biomolecule representation of the first biomolecule the first biomolecule frequency and the second biomolecule frequency; and outputting the inferred fitness score for the first biomolecule by the machine-learning model based on the processing of the biomolecule representation of the first biomolecule.

Embodiment 3: The method of Embodiment 2, wherein the machine-learning model was trained using sequencing time-series data associated with biomolecule frequencies of particular biomolecules, wherein the sequencing time-series data was obtained from directed evolution of a population of biomolecules over a plurality of rounds, and wherein the population of biomolecules in each round was a unique set of biomolecules with respect to each other round.

Embodiment 4: The method of Embodiment 3, further including: training the machine-learning model, wherein the training includes learning inferred fitness scores for the population of biomolecules by predicting biomolecule frequencies of the population of biomolecules in the respective round given, for each biomolecule in the population of biomolecules, the biomolecule frequency of the respective biomolecule in one or more prior rounds having a non-zero frequency of the respective biomolecule.

Embodiment 5: The method of Embodiment 4, wherein predicting biomolecule frequencies of the population of biomolecules in the respective round given, for each biomolecule in the population of biomolecules, the biomolecule frequency of the respective biomolecule in one or more prior rounds having a non-zero frequency of the respective biomolecule includes: determining an initial round for each biomolecule where the biomolecule has a non-zero abundance; predicting an abundance associated with each biomolecule in each round after the initial round based on the non-zero abundance; and predicting biomolecule frequencies of the population of biomolecules in the respective round given predicted abundances associated with the population of biomolecules in the respective round.

Embodiment 6: The method of either one of Embodiments 4-5, wherein the training further includes: identifying, based on the sequencing time-series data, one or more first biomolecule frequencies associated with one or more first biomolecules of the population of biomolecules that are greater than zero in a first round of the plurality of rounds; identifying, based on the sequencing time-series data, one or more second biomolecule frequencies associated with the one or more first biomolecules in a second round consecutive to the first round; predicting biomolecule frequencies associated with the one or more first biomolecules in the second round given the one or more first biomolecule frequencies; calculating a loss between the predicted biomolecule frequencies and the second biomolecule frequencies; and determining baseline fitness scores for the population of biomolecules based on the loss.

Embodiment 7: The method of any one of Embodiments 4-6, wherein learning the inferred fitness scores for the population of biomolecules is based on the baseline fitness scores for the population of biomolecules.

Embodiment 8: The method of any one of Embodiments 4-7, wherein learning the inferred fitness scores in the training of the machine-learning model includes optimizing a Dirichlet-multinomial loss function.

Embodiment 9: The method of any one of Embodiments 4-8, wherein the training of the machine-learning model further includes: calculating a Dirichlet loss negative log-likelihood between the predicted biomolecule frequencies and actual biomolecule frequencies as a negative log-likelihood.

Embodiments 10: The method of Embodiment 3, wherein the first biomolecule is within the population of biomolecules in the plurality of rounds, wherein the first biomolecule is present in a second-to-last round of the plurality of rounds, wherein the method further includes: predicting, based on the inferred fitness score for the first biomolecule, a biomolecule frequency of the first biomolecule in a last round of the plurality of rounds.

Embodiment 11: The method of either one of Embodiments 3 and 10, wherein the plurality of rounds includes at least two rounds.

Embodiment 12: The method of any one of Embodiments 3 and 11, wherein the first biomolecule is outside of the population of biomolecules in the plurality of rounds.

Embodiment 13: The method of Embodiment 2, further including: processing a plurality of biomolecule representations associated with a plurality of respective second biomolecules by the machine-learning model to determine a plurality of inferred fitness scores for the plurality of second biomolecules, respectively; and selecting, based on the inferred fitness scores for the plurality of second biomolecules and pairwise distances between the plurality of second biomolecules, one or more diverse biomolecules from the plurality of second biomolecules, and wherein the one or more diverse biomolecules meet a predetermined criteria for selection.

Embodiment 14: The method of either one of Embodiments 2 and 13, further including: processing a plurality of biomolecule representations associated with a plurality of respective second biomolecules by the machine-learning model to determine a plurality of inferred fitness scores for the plurality of second biomolecules, respectively; generating one or more clusters for the plurality of biomolecule representations based on a clustering algorithm; and selecting one or more diverse biomolecules from the plurality of second biomolecules by identifying the one or more diverse biomolecules from the one or more clusters, respectively, wherein each of the one or more diverse biomolecules is associated with a top inferred fitness score in the respective cluster.

Embodiment 15: The method of any one of Embodiments 2 and 13-14, further including: processing a plurality of biomolecule representations associated with a plurality of respective second biomolecules by the machine-learning model to determine a plurality of inferred fitness scores for the plurality of second biomolecules, respectively; and selecting, based on the inferred fitness scores for the plurality of second biomolecules, one or more second biomolecules meeting a predetermined criteria for selection, wherein one or more of the selected second biomolecules are each associated with a low relative biomolecule frequency in a last round of the plurality of rounds.

Embodiment 16: The method of any one of Embodiments 1-15, wherein the sequencing time-series data include DNA sequencing time-series data collected from one or more assays including one or more of a yeast display, a phage display, an mRNA display, a ribosomal display, a yeast growth, a deep mutational scan, a gene enrichment screen, or a selection on DNA encoded chemical libraries.

Embodiment 17: The method of any one of Embodiments 1-16, wherein the sequencing time-series data include DNA sequencing time-series data collected from a yeast display, and wherein the first biomolecule includes an antibody, wherein the method further includes: determining, based on the inferred fitness score for the antibody, that a binding affinity of the antibody to receptor tyrosine kinase meets a predetermined criteria for selection.

Embodiment 18: The method of any one of Embodiments 1-17, wherein the sequencing time-series data include DNA sequencing time-series data collected from an mRNA display, and wherein the first biomolecule includes a macrocyclic peptide, wherein the method further includes: determining, based on the inferred fitness score for the macrocyclic peptide, that a binding affinity of the macrocyclic peptide to one or more domains of receptor tyrosine kinase meets a predetermined criteria for selection.

Embodiment 19: The method of any one of Embodiments 1-18, further including: determining, based on the inferred fitness score for the first biomolecule, that a biological activity associated with the first biomolecule meets a predetermined criteria for selection.

Embodiment 20: The method of any one of Embodiments 1-19, wherein the inferred fitness score for the first biomolecule indicates a biological activity 1, wherein the sequencing time-series data include DNA sequencing time-series data, and wherein the first or second biomolecule frequency of the first biomolecule indicate genotype frequency.

Embodiment 21: The method of any one of Embodiments 1-20, wherein the sequencing time-series data comprise DNA sequencing time-series data, and wherein the first or second biomolecule frequency of the first biomolecule indicates genotype frequency.

Embodiment 22: One or more computer-readable non-transitory storage media embodying software that is operable when executed to: obtain sequencing time-series data from a first round of directed evolution, wherein the sequencing time-series data for the first round includes a first biomolecule frequency of a first biomolecule, the first biomolecule frequency of the first biomolecule being a non-zero frequency; obtain sequencing time-series data from a second round of directed evolution, wherein the sequencing time-series data for the second round includes a second biomolecule frequency of the first biomolecule; and output an inferred fitness score for the first biomolecule using the first biomolecule frequency and the second biomolecule frequency.

Embodiment 23: A system including: one or more processors; and a non-transitory memory coupled to the processors including instructions executable by the processors, the processors operable when executing the instructions to: obtain sequencing time-series data from a first round of directed evolution, wherein the sequencing time-series data for the first round includes a first biomolecule frequency of a first biomolecule, the first biomolecule frequency of the first biomolecule being a non-zero frequency; obtain sequencing time-series data from a second round of directed evolution, wherein the sequencing time-series data for the second round includes a second biomolecule frequency of the first biomolecule; and output an inferred fitness score for the first biomolecule using the first biomolecule frequency and the second biomolecule frequency.

### Miscellaneous

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

What is claimed is:

1. A method comprising, by one or more computing systems:
obtaining sequencing time-series data from a first round of directed evolution, wherein the sequencing time-series data for the first round comprises a first biomolecule frequency of a first biomolecule, the first biomolecule frequency of the first biomolecule being a non-zero frequency;
obtaining sequencing time-series data from a second round of directed evolution, wherein the sequencing time-series data for the second round comprises a second biomolecule frequency of the first biomolecule; and
outputting an inferred fitness score for the first biomolecule using the first biomolecule frequency and the second biomolecule frequency.

2. The method of claim 1, further comprising:
accessing a biomolecule representation of the first biomolecule;
processing, by a machine-learning model, the biomolecule representation of the first biomolecule based on the first biomolecule frequency and the second biomolecule frequency; and
outputting the inferred fitness score for the first biomolecule by the machine-learning model based on the processing of the biomolecule representation of the first biomolecule.

3. The method of claim 2, wherein the machine-learning model was trained using sequencing time-series data associated with biomolecule frequencies of particular biomolecules, wherein the sequencing time-series data was obtained from directed evolution of a population of biomolecules over a plurality of rounds, and wherein the population of biomolecules in each round was a unique set of biomolecules with respect to each other round.

4. The method of claim 3, further comprising:
training the machine-learning model, wherein the training comprises learning inferred fitness scores for the population of biomolecules by predicting biomolecule frequencies of the population of biomolecules in the respective round given, for each biomolecule in the population of biomolecules, the biomolecule frequency of the respective biomolecule in one or more prior rounds having a non-zero frequency of the respective biomolecule.

5. The method of claim 4, wherein predicting biomolecule frequencies of the population of biomolecules in the respective round given, for each biomolecule in the population of biomolecules, the biomolecule frequency of the respective biomolecule in one or more prior rounds having a non-zero frequency of the respective biomolecule comprises:
determining an initial round for each biomolecule where the biomolecule has a non-zero abundance;
predicting an abundance associated with each biomolecule in each round after the initial round based on the non-zero abundance; and
predicting biomolecule frequencies of the population of biomolecules in the respective round given predicted abundances associated with the population of biomolecules in the respective round.

6. The method of claim 4, wherein the training further comprises:
identifying, based on the sequencing time-series data, one or more first biomolecule frequencies associated with one or more first biomolecules of the population of biomolecules that are greater than zero in a first round of the plurality of rounds;
identifying, based on the sequencing time-series data, one or more second biomolecule frequencies associated with the one or more first biomolecules in a second round consecutive to the first round;
predicting biomolecule frequencies associated with the one or more first biomolecules in the second round given the one or more first biomolecule frequencies;
calculating a loss between the predicted biomolecule frequencies and the second biomolecule frequencies; and
determining baseline fitness scores for the population of biomolecules based on the loss.

7. The method of claim 6, wherein learning the inferred fitness scores for the population of biomolecules is based on the baseline fitness scores for the population of biomolecules.

8. The method of claim 4, wherein learning the inferred fitness scores in the training of the machine-learning model comprises optimizing a Dirichlet-multinomial loss function.

9. The method of claim 8, wherein the training of the machine-learning model further comprises:
calculating a Dirichlet loss negative log-likelihood between the predicted biomolecule frequencies and actual biomolecule frequencies as a negative log-likelihood.

10. The method of claim 3, wherein the first biomolecule is within the population of biomolecules in the plurality of rounds, wherein the first biomolecule is present in a second-to-last round of the plurality of rounds, wherein the method further comprises:
predicting, based on the inferred fitness score for the first biomolecule, a biomolecule frequency of the first biomolecule in a last round of the plurality of rounds.

11. The method of claim 3, wherein the plurality of rounds comprises at least two rounds.

12. The method of claim 3, wherein the first biomolecule is outside of the population of biomolecules in the plurality of rounds.

13. The method of claim 2, further comprising:
processing a plurality of biomolecule representations associated with a plurality of respective second biomolecules by the machine-learning model to determine a plurality of inferred fitness scores for the plurality of second biomolecules, respectively; and
selecting, based on the inferred fitness scores for the plurality of second biomolecules and pairwise distances between the plurality of second biomolecules, one or more diverse biomolecules from the plurality of second biomolecules, and wherein the one or more diverse biomolecules meet a predetermined criteria for selection.

14. The method of claim 2, further comprising:
processing a plurality of biomolecule representations associated with a plurality of respective second biomolecules by the machine-learning model to determine a plurality of inferred fitness scores for the plurality of second biomolecules, respectively;
generating one or more clusters for the plurality of biomolecule representations based on a clustering algorithm; and
selecting one or more diverse biomolecules from the plurality of second biomolecules by identifying the one or more diverse biomolecules from the one or more clusters, respectively, wherein each of the one or more diverse biomolecules is associated with a top inferred fitness score in the respective cluster.

15. The method of claim 2, further comprising:
processing a plurality of biomolecule representations associated with a plurality of respective second biomolecules by the machine-learning model to determine a plurality of inferred fitness scores for the plurality of second biomolecules, respectively; and
selecting, based on the inferred fitness scores for the plurality of second biomolecules, one or more second biomolecules meeting a predetermined criteria for selection, wherein one or more of the selected second biomolecules are each associated with a low relative biomolecule frequency in a last round of the plurality of rounds.

16. The method of claim 1, wherein the sequencing time-series data comprise DNA sequencing time-series data collected from one or more assays comprising one or more of a yeast display, a phage display, an mRNA display, a ribosomal display, a yeast growth, a deep mutational scan, a gene enrichment screen, or a selection on DNA encoded chemical libraries.

17. The method of claim 1, wherein the sequencing time-series data comprise DNA sequencing time-series data collected from a yeast display, and wherein the first biomolecule comprises an antibody, wherein the method further comprises:
determining, based on the inferred fitness score for the antibody, that a binding affinity of the antibody to receptor tyrosine kinase meets a predetermined criteria for selection.

18. The method of claim 1, wherein the sequencing time-series data comprise DNA sequencing time-series data collected from an mRNA display, and wherein the first biomolecule comprises a macrocyclic peptide, wherein the method further comprises:
determining, based on the inferred fitness score for the macrocyclic peptide, that a binding affinity of the macrocyclic peptide to one or more domains of receptor tyrosine kinase meets a predetermined criteria for selection.

19. The method of claim 1, further comprising:
determining, based on the inferred fitness score for the first biomolecule, that a biological activity associated with the first biomolecule meets a predetermined criteria for selection.

20. The method of claim 1, wherein the inferred fitness score for the first biomolecule indicates a biological activity of the first biomolecule with respect to a target protein.

21. The method of claim 1, wherein the sequencing time-series data comprise DNA sequencing time-series data, and wherein the first or second biomolecule frequency of the first biomolecule indicates genotype frequency.

22. One or more computer-readable non-transitory storage media embodying software that is operable when executed to:
obtain sequencing time-series data from a first round of directed evolution, wherein the sequencing time-series data for the first round comprises a first biomolecule frequency of a first biomolecule, the first biomolecule frequency of the first biomolecule being a non-zero frequency;
obtain sequencing time-series data from a second round of directed evolution, wherein the sequencing time-series data for the second round comprises a second biomolecule frequency of the first biomolecule; and
output an inferred fitness score for the first biomolecule using the first biomolecule frequency and the second biomolecule frequency.

23. A system comprising: one or more processors; and a non-transitory memory coupled to the processors comprising instructions executable by the processors, the processors operable when executing the instructions to:
obtain sequencing time-series data from a first round of directed evolution, wherein the sequencing time-series data for the first round comprises a first biomolecule frequency of a first biomolecule, the first biomolecule frequency of the first biomolecule being a non-zero frequency;
obtain sequencing time-series data from a second round of directed evolution, wherein the sequencing time-series data for the second round comprises a second biomolecule frequency of the first biomolecule; and
output an inferred fitness score for the first biomolecule using the first biomolecule frequency and the second biomolecule frequency.
